(19) United States
(12) Patent Application Publication
(10) Pub. No.: US 2021/0139846 A1
(43) Pub. Date: May 13, 2021

(54) ENGINEERED CELLS, AND METHODS OF USING THE SAME

(71) Applicant: Board of Supervisors of Louisiana State University and Agricultural and Mechanical College, Baton Rouge, LA (US)

(72) Inventor: Nicolas Bazan, New Orleans, LA (US)

(21) Appl. No.: 16/618,645

(22) PCT Filed: Jun. 4, 2018
(86) PCT No.: PCT/US2018/035822

§ 371 Date: Dec. 2, 2019

Related U.S. Application Data
(60) Provisional application No. 62/514,337, filed on Jun. 2, 2017.

(51) Int. Cl. C12N 5/079; C07K 14/72; A61K 35/30; A61K 31/202; A61P 25/00

(52) U.S. Cl. CPC C12N 5/0618; C07K 14/72; A61P 25/00; A61K 31/202; A61K 35/30

(57) ABSTRACT

This invention is directed towards genetically engineered cells, methods of making genetically engineered cells, and methods of using the same.

This application claims priority from U.S. Provisional Application No. 62/514,337, filed on Jun. 2, 2017, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### GOVERNMENT INTERESTS

This invention was made with government support under Grant No. R01 NS046741 and Grant No. R01 GM103340 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed towards genetically engineered cells, methods of making genetically engineered cells, and methods of using the same.

### BACKGROUND OF THE INVENTION

There is no cure for any retina or CNS disease. At the onset of these diseases, uncompensated oxidative stress triggers multiple pathways that converge on neuroinflammatory disturbances that foster cell death. Therefore there is an unmet need for effective therapies that at least would slow down the onset of retinal degenerative disease (retinitis pigmentosa, age-related macular degeneration) and of neurodegenerative disease such as Alzheimer's, Parkinson's, MS, ALS and others.

### SUMMARY OF THE INVENTION

The present invention provides a genetically engineered cell line comprising a plurality of cells transformed with at least one polynucleotide encoding a polypeptide, wherein the expressed polypeptide transports an Omega-3 fatty acid and/or a long chain fatty acid across the cell membrane. In embodiments, the cell line can be cryopreserved, so as to maintains its efficacy and/or viability. Embodiments comprise a cryopreserved composition for the treatment of diseases, non-limiting examples of which comprise Alzheimer's disease, epilepsy, Parkinson's disease, stroke, Huntington's disease, traumatic brain injury, spinal cord injury, Amyotrophic lateral scelerosis, and age-related macular degeneration.

In embodiments, the polynucleotides comprises DNA, RNA, or a fragment thereof. In embodiments, the polynucleotide comprises a synthetic polynucleotide.

In embodiments, the the polynucleotide is about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1.

In embodiments, the the polynucleotide is about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2.

In embodiments, the polynucleotide stably integrates into the genome of the cell.

In embodiments, the plurality of cells comprise skin fibroblasts, adipose tissue stem cells, primary retinal pigment epithelial cells, human adult stem cells, transdifferentiated neuronal cells, pericytes, or macrophages.

In embodiments, the plurality of cells are isolated from skin, adipose tissue, bone marrow, blood, brain tissue, ocular tissue, or a combination thereof.

In embodiments, the polypeptide comprises Adiponectin receptor 1 or a fragment thereof, membrane-type Frizzled Related Protein or a fragment thereof; or a combination thereof. For examples, in embodiments, the polypeptide is about 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3, SEQ ID NO: 4, SEQ ID NO: 5, SEQ ID NO: 6, and/or SEQ ID NO: 7.

In embodiments, the omega-3 fatty acid comprises docosahexaenoic acid (DHA) and eicosapentaenoic acid (EPA), or other long-chain fatty acids.

Embodiments further comprise a method of generating the genetically engineered cell line as described herein. For example, embodiments comprise obtaining a plurality of cells, and introducing into the plurality of cells at least one polynucleotide encoding a polypeptide, wherein the expressed polypeptide transports an Omega-3 fatty acid across the cell membrane.

Embodiments further comprise a method of culturing the genetically engineered cells, so as to produce a population of cells comprising genetically engineered cells.

Further, embodiments comprise an in vitro culture of genetically engineered cells as described herein.

Embodiments can further comprise detecting the presence of the polypeptide within the plurality of cells, for example by FACS or immunohistochemistry, such as Western blot analysis.

In embodiments, the plurality of cells comprise stem cells, skin fibroblasts, adipose tissue stem cells, primary retinal pigment epithelial cells, human adult stem cells, transdifferentiated neuronal cells, pericytes, macrophages, or a combination thereof.

In embodiments, the plurality of cells are isolated from adipose tissue, bone marrow, blood, brain tissue, ocular tissue, or a combination of the cells listed herein.

Embodiments are further directed towards a therapeutic composition comprising a plurality of genetically engineered cells transformed with at least one polynucleotide encoding a polypeptide, wherein the expressed polypeptide transports an Omega-3 fatty acid and/or a long chain fatty acid across the cell membrane, and a pharmaceutically acceptable carrier. Optionally, the composition can further comprise at least one omega-3 fatty acid, non-limiting examples of which include docosahexaenoic acid (DHA) and EPA.

Embodiments are further directed towards a conditioned media possessing the biological property of being neuroprotective. For example, the conditioned media is characterized by being the product of culturing a plurality of genetically engineered cells as described herein for a period of time. For example, the culturing comprises a period of time of about 1 day, 2 days, 3 days, 4 days, 5 days, 6 day, 7 days, 2 weeks, 3 weeks, or 1 month.

In embodiments, the cells are removed from the media after the culturing period.

In embodiments, the cells secrete neuroprotective factors into the conditioned medium. Non-limiting examples of the neuroprotective protective factors comprise cytokines, such as IL-10; lipid mediators, such as NPD1, elovanoids, or docosanoids; exosomes, such as those derived from stem cells, or a combination thereof.

In embodiments, the conditioned media is cytoprotective and/or neuroprotective. For example, the conditioned media can be administered to a subject so as to treat a subject afflicted with a neurological disease.

Embodiments further comprise a method of reducing or ameliorating symptoms associated with a neurological disease, a method of reducing or ameliorating pathogenesis associated with a neurological disease, a method of delaying the onset and/or progression of a neurological disease, or a method of promoting neuronal recovery and restoration of function. For example, the methods comprise administering to a subject genetically engineered cells transformed with at least one polynucleotide encoding a polypeptide, wherein the expressed polypeptide transports an Omega-3 fatty acid and/or a long chain fatty acid across the cell membrane and related compositions. Optionally, embodiments can further comprise administering to the subject an Omega-3 fatty acid or other long-chain fatty acid, such as DHA or EPA.

In embodiments, neuronal recovery and/or restoration of function can be measured by EEG or neurological assessment.

In embodiments, at least one of the plurality of genetically engineered cells engrafts within a tissue of the nervous system of the subject after administration to the subject. For example, the nervous system comprises the central nervous system, the peripheral nervous system. For example, the tissue comprises the brain, spinal cord, optic nerve or combination thereof.

Embodiments as described herein can be used to treat, prevent, reverse and/or delay the onset of symptoms and/or pathogenesis of diseases such as neurological disease. Such diseases can be associated with neuroinflammation, neuronal cell death, neuronal cell injury, or a combination thereof.

In embodiments, the neurological disease comprises a neurodegenerative disease, non-limiting examples of which comprise Alzheimer's disease, epilepsy, Parkinson's disease, stroke, Huntington's disease, traumatic brain injury, spinal cord injury, Amyotrophic lateral scelerosis, and age-related macular degeneration.

Non-limiting examples of disease pathogenesis that can be treated, prevented, delayed, and/or reversed include neuroinflammation, neuronal death, neuronal injury, Aβ formation, infarct volume, and/or oxidative stress.

Embodiments as described herein can promote neuronal survival, neuronal differentiation, neurogenesis, neurite growth, synaptogenesis, synaptic protein expression, synaptic function, or a combination thereof. For example, neurogenesis can be detected using 5-bromo-2′-deoxyuridine, Ki-67, DCX, NeuN, or a combination thereof.

Embodiments further comprise a kit, such as that comprising a plurality of genetically engineered cells as described herein, the therapeutic compositions as described herein, the conditioned media as described herein, or a combination thereof, and instructions for use. For example, the plurality of genetically engineered cells, the therapeutic compositions, the conditioned media, or a combination thereof can be provided in a vessel.

Other objects and advantages of this invention will become readily apparent from the ensuing description.

### BRIEF DESCRIPTION OF THE FIGURES

FIG. 1 shows the pathway by which genetically engineered cells enhances DHA delivery to cells and in turn activates the conversion of the fatty acid into bioactive docosanoid.

FIG. 2 shows the pathway by which genetically engineered cells enhances DHA delivery to cells and in turn activates the conversion of the fatty acid into bioactive docosanoids that elicit multiple pro-homeostatic actions.

FIG. 3 shows details of embodiments of the invention.

FIG. 4 shows schematic detailing methods of making embodiments of the invention.

FIG. 5 shows genetically-engineered cells will foster homeostasis:molecular sensors that counteract homeostasis disruptions in the Central Nervous System.

FIG. 6 shows genetically-engineered cells will sustain reversal of A-beta peptide formation in Alzheimer's Disease and Age-Related Macular Degeneration.

FIG. 7 demonstrates that neuroprotection occurs at 7 days when DHA is administered in the identical site where the GESC would be administered. DHA treatment reduced ischemic brain damage (A, C) and improves behavioral functions (B). Without wishing to be bound by theory, GESCs will have a more beneficial and longer lasting effect than DHA alone. Under these conditions, the injected DHA only protects some of the damaged cells but does not induce neurorestoration because it does not have the properties of the GESC to generate new cells to replace those cells that were lost, or the ability to capture DHA (which leads to the synthesis of prohomeostatic docosanoids) due to the expression of the selected proteins.

FIG. 8 shows immunofluorescence analysis of six-day-old transdifferentiated neurons from skin fibroblasts which demonstrate coexpression of synaptic marker synaptophysin I and of neuronal specific nuclear marker (NeuN).

FIG. 9 shows a Western blot analysis of adipose tissue-derived stem cells, transduced with lentivirus containing AdipoR1 overexpression cDNA in tandem with mCherry under the strong CMV promotor. This results in a robust (>2 fold) increase in AdipoR1 expression, compared to lentivirus with mCherry alone.

FIG. 10 shows a diagram of experiments to be completed. Stem cells (SF or AT) will be injected into the ipsilesional lateral cerebral ventricle and DHA will be iv injected, both on day 1 after 2 hours of ischemic stroke.

FIG. 11 demonstrates that human adipose tissue-derived stem cells (hASCs) overexpressing AdipoR1 show greater increase in specific DHA uptake as determined by LC-MS/MS. Stable clones of hASCs overexpressing AdipoR1 or mCherry following lentivirus transduction, were incubated overnight in the presence or not of DHA-d5 or AA-d8 (1 μM) and vehicle DMSO with various concentrations of AdipoR1 antagonist adiporon (1-50 μM). Compared to mCherry and in the absence of agonist AdipoR1 transduced cells showed significant 3 times increase in DHA-d5 uptake; this effect was further increased by the addition of adiporon. Compared to DHA-d5 uptake, levels of AA-d8 were extremely low under these conditions.

FIG. 12 demonstrates DHA accelerates chemical transdifferentiation of human adipose tissue derived stem cells into neurons. Images were taken 24 hours following incubation in the presence of induction media containing or not containing DHA (504). Similar to skin derived fibroblasts, DHA strongly increased the percentage of morphologically mature neurons (blue arrow) compared to induction medium alone.

FIG. 13 shows immunofluorescence analysis of stem cells and neurons. Transdifferentiated neuronal stem cells and human skin fibroblasts co-transduced with lentivirus to induce overexpression of (A) mCherry-GFP or (B) AdipoR1-mCherry/MFRP-GFP. Expression of neuronal-type biomarkers by the rat primary neurons.

FIG. 14 shows baseline levels of neuronal cultures cell density with the alamarBlue assay. Fluorescene intensities for the alamarBlue assay with the molecular devices M5 plate reader (excitation 540 nm/emission 590 nm) were converted into percent cells by dividing the actual fluorescence against the mean fluorescence of the untreated control cells×100. Student's t-test was used to determine statistical significance p value set at p≤0.05. (A) Cortical neurons, (B) hippocampal neurons.

FIG. 15 shows three days after OGD: density of neuronal cultures assesseb by the alamarBlue assay. (A) cortical neurons, (B) hippocampal neurons. Statistical analysis (*): p≤0.05 vs untreated control; (#): p≤0.05 vs OGD-BM-mCherry-GFP; (&): p≤0.05 vs OGD-Ins-BM-mCherry-GFP.

FIG. 16 shows DHA improved total neurologic deficits. SD male rats

(11-12 weeks old); Model: 2 h of MCAo by intraluminal suture; DHA (5 mg/kg) or Saline treatments (IV)—at 3 h from onset of MCAo; BrdU labeling at days 4, 5, 6; Immunohistochemistry on week 2

FIG. 17 shows immunohistochemistry with BrdU/ki-67.

FIG. 18 shows immunohistochemistry with BrdU/ki-67. DHA increased BrdU⁺/ki-67⁺ cells in cortex, SVZ and DG.

FIG. 19 shows immunohistochemistry with BrdU/DCX.

FIG. 20 shows immunohistochemistry with BrdU/DCX. DHA increased BrdU⁺/DCX⁺ cells in cortex, SVZ and DG.

FIG. 21 shows immunohistochemistry with BrdU/NeuN.

FIG. 22 shows immunohistochemistry with BrdU/NeuN. DHA increased BrdU⁺/NeuN⁺ cells in cortex, SVZ and DG

FIG. 23 shows histopathology. DHA reduced cortical and total infarct volume.

FIG. 24 shows the vector information for EX-W0161-Lv103.

FIG. 25 shows the vector information for EX-Z1681-Lv111.

### DETAILED DESCRIPTION OF THE INVENTION

There is no cure for any retina or CNS disease. At the onset of these diseases, uncompensated oxidative stress triggers multiple pathways that converge on neuroinflammatory disturbances that foster cell death. Therefore there is an unmet need for effective therapies that at least would slow down the onset of retinal degenerative disease (retinitis pigmentosa, age-related macular degeneration) and of neurodegenerative diseases such as Alzheimer's, Parkinson's, MS, ALS and others.

Embodiments as described herein confront this problem and are directed towards genetically engineered cells and methods of making genetically engineered cells. For example, embodiments express one or more transgenes encoding at least one protein or polypeptide, such as AdipoR, MFRP, and/or fragments thereof, implicated in the transport of an omega-3 fatty acid, such as docosahexaenoic acid, across a cellular membrane and retention of the same within a cell. For example, peptide fragments can comprise functional fragements, such as functional domains, of AdipoR or MFRP. Embodiments are directed towards compositions and kits comprising the genetically engineered cells, and methods of treating disease by administering a plurality of the genetically engineered cells or compositions comprising the same to a subject diagnosed with disease, such as that of the retina and/or CNS. Embodiments are also directed towards a cytoprotective conditioned medium that comprises neuroprotective factors, and methods of making and using the same.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

The discussion of the background to the invention herein is included to explain the context of the present invention. This is not to be taken as an admission that any of the material referred to was published, known, or part of the common general knowledge in any country as of the priority date of any of the claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein can have the same meaning as commonly understood by one of ordinary skill in the art to which this disclosure belongs. Any methods and materials similar or equivalent to those described herein can also be used in the practice or testing of the present disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### Abbreviations and Definitions

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it can modify that range by extending the boundaries above and below the numerical values set forth. In general, the term “about” is used herein to modify a numerical value above and below the stated value by a variance of 20 percent up or down (higher or lower).

The terms “sufficient” and “effective”, as used interchangeably herein, can refer to an amount (e.g. mass, volume, dosage, concentration, and/or time period) needed to achieve one or more desired result(s).

The term “administration” can refer to introducing a composition of the present disclosure into a subject. For example, one route of administration of the composition is intracranial administration. As another example, the composition can be administered by intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, “treat”, “treatment”, and/or “treating” can refer to acting upon a condition (e.g. inflammation), a disease or a disorder with a composition to affect the condition (e.g., inflammation), disease or disorder by improving or altering it. The improvement or alteration can include an improvement in symptoms or an alteration in the physiologic pathways associated with the condition (e.g., inflammation), disease, or disorder. “Treatment” can refer to one or more treatments of the disease or condition in a subject (e.g., a mammal, typically a human or non-human animal of veterinary interest), and can include: (a) reducing the risk of occurrence in a subject determined to be predisposed to the condition or disease but not yet diagnosed with it (b) impeding the development of the condition or disease, and/or (c) relieving the condition or disease, e.g., causing regression of the condition or disease and/or relieving one or more condition or disease symptoms. As used herein, the terms “prophylactically treat” or “prophylactically treating” can refer to completely or partially preventing (e.g., about 50% or more, about 60% or more, about 70% or more, about 80% or more, about 90% or more, about 95% or more, or about 99% or more) a condition (e.g., condition or disease), a disease, or a symptom thereof and/or can be therapeutic in terms of a partial or complete cure for a condition (e.g., condition or disease), a disease, and/or adverse effect attributable to the disease.

As used herein, “therapeutic” can refer to curing or treating a symptom of a disease or condition.

As used herein, the term “subject,” or “patient,” can include humans and mammals (e.g., mice, rats, pigs, cats, dogs, and horses), and non-mammals (e.g., ayes such as chickens etc.). Typical subjects to which compounds of the present disclosure can be administered will be mammals, particularly primates, especially humans. For veterinary applications, a wide variety of subjects will be suitable, non-limiting examples of which comprise livestock such as cattle, sheep, goats, cows, swine; poultry such as chickens, ducks, geese, turkeys; and domesticated animals particularly pets such as dogs and cats. For diagnostic or research applications, a wide variety of mammals can be suitable subjects, non-limiting examples of which comprise rodents (e.g., mice, rats, hamsters), rabbits, primates, and swine such as inbred pigs and the like. The term “living subject” can refer to a subject noted above or another organism that is alive.

As used herein, the terms “peptide”, “polypeptide” and “protein” are used interchangeably herein. Unless otherwise clear from the context, the noted terms can refer to a polymer having at least two amino acids linked through peptide bonds, non-limiting examples of which comprise oligopeptides, protein fragments, such as functional domains, glycosylated derivatives, pegylated derivatives, fusion proteins and the like.

Before explaining at least one embodiment of the disclosure in detail, it is to be understood that the disclosure is not necessarily limited in its application to the details set forth in the following description or exemplified by the examples. The disclosure is capable of other embodiments or of being practiced or carried out in various ways. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Transgenes

As used herein, the term “transgene” or “(trans)gene” can refer to a particular nucleic acid sequence encoding a polypeptide or a portion of a polypeptide to be expressed in a cell into which the nucleic acid sequence is inserted. For example, the polypeptide can comprise AdipoR, MFRP, and/or fragments thereof. In embodiments, the polypeptide comprises AdipoR, MFRP, and/or fragments thereof. For example, the polypeptide comprises SEQ ID 1, SEQ ID 2, SEQ ID 3, SEQ ID 4, SEQ ID 5, SEQ ID 6, or SEQ ID 7. However, it is also possible that transgenes are expressed as RNA, typically to lower the amount of a particular polypeptide in a cell into which the nucleic acid sequence is inserted. These RNA molecules include but are not limited to molecules that exert their function through RNA interference (shRNA, RNAi), micro-RNA regulation (miRNA), catalytic RNA, antisense RNA, RNA aptamers, etc. For example, the nucleic acid can be introduced into a cell through integration in the genome or as an episomal plasmid. Integration can be stable integration into the genome, for example. Of note, expression of the transgene can be restricted to a subset of the cells into which the nucleic acid sequence is inserted (Sambrook et al., Molecular Cloning: A Laboratory Manual, Cold SpringHarbor Laboratory, Cold Spring Harbor, 1989). The term ‘transgene’ can refer to (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced. “Mutant form” can refer to a nucleic acid sequence that contains one or more nucleotides that are different from the wild-type or naturally occurring sequence (i.e., the mutant nucleic acid sequence contains one or more nucleotide substitutions, deletions, and/or insertions). In some cases, the transgene can also include a sequence encoding a leader peptide or signal sequence such that the transgene product will be secreted from the cell.

The invention is directed towards a genetically engineered cell line that is transformed with at least one polynucleotide or transgene encoding for a polypeptide and/or protein that transports an omega-3 fatty acid across the cell membrane. For example, the protein can comprise the cell-membrane bound proteins, AdipoR1 (such as AdipoR1 human variant 4; Accession No. NM_001290553; SEQ ID 3, or mouse AdipoR1, SEQ ID 4), MFRP (such as human MFRP, Accession No. NM_031433; SEQ ID 5; or mouse MFRP, Accession No. NM_001190314; SEQ ID 6 and/or SEQ ID 7), or both AdipoR1 and MFRP Adiponectin Receptor 1 protein (AdipoR1) and/or Membrane-type Frizzled Related Protein (MFRP), or polypeptide fragments thereof.

For example, the invention is directed towards a genetically engineered cell line that is transformed with at least one polynucleotide or transgene encoding for a polypeptide and/or protein that transports an omega-3 fatty acid across the cell membrane, wherein the polypeptide, protein, or fragment thereof comprises one or more of SEQ ID 1, SEQ ID 2, SEQ ID 3, SEQ ID 4, SEQ ID 5, SEQ ID 6, or SEQ ID 7.

The nucleotide sequence of Homo sapiens membrane frizzled-related protein (MFRP) (SEQ ID 1) is:

[symbol]

AAGGACTTCTCAGATGTCATCCTCTGCA
TGGAGGCAACAGAATCGAGCAAGACCGAGTTCTGCAATCCTGCCTTCG
AGCCTGAGTCTGGGCCACCCTGCCCTCCCCCAGTTTTCCCAGAGGATG
CCAGCTACAGCGTCCCAGCTCCCTGGCATGGTCGGCGTCCTCGAGGGC
TACGGCCAGACTGCCGCTTCTCCTGGCTCTGTGTCCTCCTGCTCTCCA
GCCTGCTCCTCCTGCTGCTTGGGCTGCTGGTGGCCATCATCCTGGCCC
AGCTGCAGGCTGCACCCCCATCTGGGGCGTCCCATAGCCCACTGCCTG
CCGGAGGCCTTACCACGACCACCACCACCCCCACCATCACCACCTCTC
AGGCAGCTGGGACCCCTAAAGGGCAGCAGGAGTCAGGCGTGAGCCCCT
CCCCACAGTCCACCTGTGGAGGCCTCCTCTCTGGCCCAAGGGGCTTCT
TCAGCAGCCCTAACTACCCAGACCCTTACCCCCCCAACACCCACTGCG
TGTGGCATATCCAGGTGGCCACAGACCATGCAATACAGCTCAAGATCG
AAGCCCTCAGCATAGAGAGTGTGGCCTCTTGCCTTTTTGATCGCTTGG
AACTCTCCCCTGAGCCTGAAGGCCCCCTCCTCAGGGTTTGTGGAAGGG
TGCCTCCCCCCACGCTCAACACCAATGCCAGCCACCTCCTGGTGGTCT
TCGTCTCTGACAGCAGTGTGGAAGGATTTGGTTTCCATGCCTGGTACC
AGGCTATGGCCCCTGGGCGCGGGAGCTGTGCCCATGATGAGTTCCGCT
GTGACCAGCTCATCTGCCTGCTACCTGACTCAGTGTGTGATGGTTTTG
CCAACTGTGCTGACGGCAGTGATGAGACCAATTGCAGTGCCAAGTTCT
CGGGGTGTGGGGGGAATCTGACTGGCCTCCAGGGCACTTTCTCTACTC
CCAGCTACCTGCAGCAGTACCCTCACCAACTGCTCTGCACCTGGCATA
TCTCGGTGCCTGCCGGACACAGCATAGAACTACAGTTCCACAACTTCA
GCCTGGAGGCTCAGGACGAGTGCAAGTTTGACTACGTGGAGGTGTATG
AGACCAGCAGCTCAGGGGCCTTCAGCCTCCTGGGCAGGTTCTGTGGAG
CAGAGCCACCCCCCCACCTCGTCTCCTCGCACCATGAGCTGGCTGTGC
TGTTTAGGACAGATCATGGCATCAGCAGTGGAGGCTTCTCAGCCACCT
ACCTGGCCTTCAATGCCACGGAGAACCCCTGTGGGCCCAGTGAGCTCT
CCTGCCAGGCAGGAGGGTGTAAGGGTGTGCAGTGGATGTGTGACATGT
GGAGAGACTGCACCGATGGCAGCGATGACAACTGCAGCGGCCCCTTGT
TCCCACCCCCAGAGCTGGCCTGTGAGCCTGTCCAGGTGGAGATGTGCC
TCGGTCTGAGCTACAACACCACAGCCTTCCCTAACATCTGGGTGGGCA
TGATCACCCAGGAGGAGGTGGTAGAGGTCCTCAGCGGTTACAAGAGCC
TGACAAGCCTGCCCTGCTACCAGCATTTCCGGAGGCTCCTGTGTGGGC
TGCTTGTGCCCCGTTGCACCCCACTAGGCAGTGTTCTGCCCCCTTGCC
GCTCTGTCTGCCAGGAAGCGGAGCACCAGTGCCAGTCTGGCCTGGCAC
TACTGGGCACCCCCTGGCCCTTCAACTGCAACAGGCTGCCAGAGGCAG
CTGACCTGGAAGCTTGTGCCCAGCCC

[symbol]

The nucleotide sequence of Homo sapiens adiponectin receptor 1 (ADIPOR1) (SEQ ID 2) is:

[symbol]

TCTTCCCACAAAGGATCTGTGGTGGCAC
AGGGGAATGGGGCTCCTGCCAGTAACAGGGAAGCTGACACGGTGGAA
CTGGCTGAACTGGGACCCCTGCTAGAAGAGAAGGGCAAACGGGTAAT
CGCCAACCCACCCAAAGCTGAAGAAGAGCAAACATGCCCAGTGCCCC
AGGAAGAAGAGGAGGAGGTGCGGGTACTGACACTTCCCCTGCAAGCC
CACCACGCCATGGAGAAGATGGAAGAGTTTGTGTACAAGGTCTGGGA
GGGACGTTGGAGGGTCATCCCATATGATGTGCTCCCTGACTGGCTAA
AGGACAACGACTATCTGCTACATGGTCATAGACCTCCCATGCCCTCC
TTTCGGGCTTGCTTCAAGAGCATCTTCCGCATTCATACAGAAACTGG
CAACATCTGGACCCATCTGCTTGGTTTCGTGCTGTTTCTCTTTTTGG
GAATCTTGACCATGCTCAGACCAAATATGTACTTCATGGCCCCTCTA
CAGGAGAAGGTGGTTTTTGGGATGTTCTTTTTGGGTGCAGTGCTCTG
CCTCAGCTTCTCCTGGCTCTTTCACACCGTCTATTGTCATTCAGAGA
AAGTCTCTCGGACTTTTTCCAAACTGGACTATTCAGGGATTGCTCTT
CTAATTATGGGGAGCTTTGTCCCCTGGCTCTATTATTCCTTCTACTG
CTCCCCACAGCCACGGCTCATCTACCTCTCCATCGTCTGTGTCCTGG
GCATTTCTGCCATCATTGTGGCGCAGTGGGACCGGTTTGCCACTCCT
AAGCACCGGCAGACAAGAGCAGGCGTGTTCCTGGGACTTGGCTTGAG
TGGCGTCGTGCCCACCATGCACTTTACTATCGCTGAGGGCTTTGTCA
AGGCCACCACAGTGGGCCAGATGGGCTGGTTCTTCCTCATGGCTGTG
ATGTACATCACTGGAGCTGGCCTTTATGCTGCTCGAATTCCTGAGCG
CTTCTTTCCTGGAAAATTTGACATATGGTTCCAGTCTCATCAGATTT
TCCATGTCCTGGTGGTGGCAGCAGCCTTTGTCCACTTCTATGGAGTC
TCCAACCTTCAGGAATTCCGTTACGGCCTAGAAGGCGGCTGTACTGA
TGACACCCTTCTC

[symbol]

[symbol]

The amino acid sequence of adiponectin receptor protein 1 (Homo sapiens; NCBI Reference Sequence: NP_001277558.1; SEQ ID 3) is:

| | |
|---|---|
| 01 | msshkgsvva qgngapasnr eadtvelael gplleekgkr vianppkaee eqtcpvpqee |
| 61 | eeevrvltlp lqahhamekm eefvykvweg rwrvipydvl pdwlkdndyl lhghrppmps |
| 121 | fracfksifr ihtetgniwt hllgfvlflf lgiltmlrpn myfmaplqek vvfgmfflga |
| 181 | vlclsfswlf htvychsekv srtfskldys giallimgsf vpwlyysfyc spqprliyls |
| 241 | ivcvlgisai ivaqwdrfat pkhrqtragv flglglsgvv ptmhftiaeg fvkattvgqm |
| 301 | gwfflmavmy itgaglyaar iperffpgkf diwfqshqif hvlvvaaafv hfygvsnlqe |
| 361 | frygleggct ddtll |

The amino acid sequence of adiponectin receptor protein 1 [Mus musculus; NCBI Reference Sequence: NP_001292998.1; SEQ ID 4) is:

| | |
|---|---|
| 1 | msshkgsaga qgngapsgnr eadtvelael gplleekgkr aasspakaee dqacpvpqee |
| 61 | eeevrvltlp lqahhamekm eefvykvweg rwrvipydvl pdwlkdndyl lhghrppmps |
| 121 | fracfksifr ihtetgniwt hllgfvlflf lgiltmlrpn myfmaplqek vvfgmfflga |
| 181 | vlclsfswlf htvychsekv srtfskldys giallimgsf vpwlyysfyc spqprliyls |
| 241 | ivcvlgisai ivaqwdrfat pkhrqtragv flglglsgvv ptmhftiaeg fvkattvgqm |
| 301 | gwfflmavmy itgaglyaar iperffpgkf diwfqshqif hvlvvaaafv hfygvsnlqe |
| 361 | frygleggct ddsll |

The amino acid sequence of membrane-type frizzled-related protein MFRP [Homo sapiens; GenBank: BAB39771.1; SEQ ID 5) is:

| | |
|---|---|
| 1 | mkdfsdvilc meatesskte fcnpafepes gppcpppvfp edasysvpap whgrrprglr |
| 61 | pdcrfswlcv lllsslllll lgllvaiila qlqaappsga shsplpaggl ttttttptit |
| 121 | tsqaagtpkg qqesgvspsp qstcggllsg prgffsspny pdpyppnthc vwhiqvatdh |
| 181 | aiqlkieals iesvasclfd rlelspepeg pllrvcgrvp pptlntnash llvvfvsdss |
| 241 | vegfgfhawy qamapgrgsc andefrcdql icllpdsvcd gfancadgsd etncsakfsg |
| 301 | cggnltglqg tfstpsylqq yphqllctwh isvpaghsie lqfhnfslea qdeckfdyve |
| 361 | vyetsssgaf sllgrfcgae ppphlvsshh elavlfrtdh gissggfsat ylafnatenp |
| 421 | cgpselscqa ggckgvqwmc dmwrdctdgs ddncsgplfp ppelacepvq vemclglsyn |
| 481 | ttafpniwvg mitqeevvev lsgyksltsl pcyqhfrrll cgllvprctp lgsvlppers |
| 541 | vcqeaehqcq sglallgtpw pfncnrlpea adleacaqp |

The amino acid sequence of membrane frizzled-related protein isoform 2 [Mus musculus; NCBI Reference Sequence: NP_001177243.1; SEQ ID 6) is:

| | |
|---|---|
| 1 | mkdyddvilr peaselskte fcnpafdpea gpscpppalq rdvgsrlqap whaqrlrglq |
| 61 | pdchfswfci lllsglllll lgllvavila qlqatslprt tknplltrgl tpmgvipstt |
| 121 | pntttttttt tpartgqqea amspthqttc ggllpgpsgf fsspnypdly pplshcvwhi |
| 181 | qvaagqtiql kiqalsiesm ltclfdrlei iseptgpllr vcgktppatl ntntshlrvs |
| 241 | fvsdndvegs gfqawyqava pghwscahne fhcdlllclk rdsvcdgite cadgsdeanc |
| 301 | saktlgcggn ltglygvfst pnypqhyphq qlctwyievp vgygirlefh nfsleaqaec |
| 361 | kfdyvevyea snlgtfsflg rfcgaeppln vvssmhqlav ifktdlgiss ggflatyqai |
| 421 | nttekfcqsg gyrdlqwmcd lwkdcandsn dncsshlspq pdltcepvqv emclglsynt |
| 481 | tafpniwvgl atqtevtdil rgyksltslp cyqtfqrflc gllvprctsl gtilppcrsv |
| 541 | cqaaeqqcqs slallgtpwp fncnrlpvaa sleacsqp |

The amino acid sequence of membrane frizzled-related protein isoform 1 [Mus musculus; NCBI Reference Sequence: NP_667337.1; SEQ ID 7) is:

| | |
|---|---|
| 1 | mkdyddvilr peaselskte fcnpafdpea gpscpppalq rdvgsrlqap whaqrlrglq |
| 61 | pdchfswfci lllsglllll lgllvavila qlqatslprt tknplltrgl tpmgvipstt |
| 121 | pntttttttt tpartgqqea amspthqttc ggllpgpsgf fsspnypdly pplshcvwhi |
| 181 | qvaagqtiql kiqalsiesm ltclfdrlei iseptgpllr vcgktppatl ntntshlrvs |
| 241 | fvsdndvegs gfqawyqava pghwscahne fhcdlllclk rdsvcdgite cadgsdeanc |
| 301 | saktlgcggn ltglygvfst pnypqhyphq qlctwyievp vgygirlefh nfsleaqaec |
| 361 | kfdyvevyea snlgtfsflg rfcgaeppln vvssmhqlav ifktdlgiss ggflatyqai |
| 421 | nttesgcpwa efcqsggyrd lqwmcdlwkd candsndncs shlspqpdlt cepvqvemcl |
| 481 | glsynttafp niwvglatqt evtdilrgyk sltslpcyqt fqrflcgllv prctslgtil |
| 541 | ppersvcqaa eqqcqsslal lgtpwpfncn rlpvaaslea csqp |

The nucleotide sequence of Homo sapiens membrane frizzled-related protein (MFRP) (SEQ ID 8) is:

AAGGACTTCTCAGATGTCATCCTCTGCATGGAGGCAACAGAATCGAGCAA
GACCGAGTTCTGCAATCCTGCCTTCGAGCCTGAGTCTGGGCCACCCTGCC
CTCCCCCAGTTTTCCCAGAGGATGCCAGCTACAGCGTCCCAGCTCCCTGG
CATGGTCGGCGTCCTCGAGGGCTACGGCCAGACTGCCGCTTCTCCTGGCT
CTGTGTCCTCCTGCTCTCCAGCCTGCTCCTCCTGCTGCTTGGGCTGCTGG
TGGCCATCATCCTGGCCCAGCTGCAGGCTGCACCCCCATCTGGGGCGTCC
CATAGCCCACTGCCTGCCGGAGGCCTTACCACGACCACCACCACCCCCAC
CATCACCACCTCTCAGGCAGCTGGGACCCCTAAAGGGCAGCAGGAGTCAG
GCGTGAGCCCCTCCCCACAGTCCACCTGTGGAGGCCTCCTCTCTGGCCCA
AGGGGCTTCTTCAGCAGCCCTAACTACCCAGACCCTTACCCCCCCAACAC
CCACTGCGTGTGGCATATCCAGGTGGCCACAGACCATGCAATACAGCTCA
AGATCGAAGCCCTCAGCATAGAGAGTGTGGCCTCTTGCCTTTTTGATCGC
TTGGAACTCTCCCCTGAGCCTGAAGGCCCCCTCCTCAGGGTTTGTGGAAG
GGTGCCTCCCCCCACGCTCAACACCAATGCCAGCCACCTCCTGGTGGTCT
TCGTCTCTGACAGCAGTGTGGAAGGATTTGGTTTCCATGCCTGGTACCAG
GCTATGGCCCCTGGGCGCGGGAGCTGTGCCCATGATGAGTTCCGCTGTGA
CCAGCTCATCTGCCTGCTACCTGACTCAGTGTGTGATGGTTTTGCCAACT
GTGCTGACGGCAGTGATGAGACCAATTGCAGTGCCAAGTTCTCGGGGTGT
GGGGGGAATCTGACTGGCCTCCAGGGCACTTTCTCTACTCCCAGCTACCT
GCAGCAGTACCCTCACCAACTGCTCTGCACCTGGCATATCTCGGTGCCTG
CCGGACACAGCATAGAACTACAGTTCCACAACTTCAGCCTGGAGGCTCAG
GACGAGTGCAAGTTTGACTACGTGGAGGTGTATGAGACCAGCAGCTCAGG
GGCCTTCAGCCTCCTGGGCAGGTTCTGTGGAGCAGAGCCACCCCCCCACC
TCGTCTCCTCGCACCATGAGCTGGCTGTGCTGTTTAGGACAGATCATGGC
ATCAGCAGTGGAGGCTTCTCAGCCACCTACCTGGCCTTCAATGCCACGGA
GAACCCCTGTGGGCCCAGTGAGCTCTCCTGCCAGGCAGGAGGGTGTAAGG
GTGTGCAGTGGATGTGTGACATGTGGAGAGACTGCACCGATGGCAGCGAT
GACAACTGCAGCGGCCCCTTGTTCCCACCCCCAGAGCTGGCCTGTGAGCC
TGTCCAGGTGGAGATGTGCCTCGGTCTGAGCTACAACACCACAGCCTTCC
CTAACATCTGGGTGGGCATGATCACCCAGGAGGAGGTGGTAGAGGTCCTC
AGCGGTTACAAGAGCCTGACAAGCCTGCCCTGCTACCAGCATTTCCGGAG
GCTCCTGTGTGGGCTGCTTGTGCCCCGTTGCACCCCACTAGGCAGTGTTC
TGCCCCCTTGCCGCTCTGTCTGCCAGGAAGCGGAGCACCAGTGCCAGTCT
GGCCTGGCACTACTGGGCACCCCCTGGCCCTTCAACTGCAACAGGCTGCC
AGAGGCAGCTGACCTGGAAGCTTGTGCCCAGCCC

The nucleotide sequence of Homo sapiens adiponectin receptor 1 (ADIPOR1) (SEQ ID 9) is:

TCTTCCCACAAAGGATCTGTGGTGGCACAGGGGAATGGGGCTCCTGCCAG
TAACAGGGAAGCTGACACGGTGGAACTGGCTGAACTGGGACCCCTGCTAG
AAGAGAAGGGCAAACGGGTAATCGCCAACCCACCCAAAGCTGAAGAAGAG
CAAACATGCCCAGTGCCCCAGGAAGAAGAGGAGGAGGTGCGGGTACTGAC
ACTTCCCCTGCAAGCCCACCACGCCATGGAGAAGATGGAAGAGTTTGTGT
ACAAGGTCTGGGAGGGACGTTGGAGGGTCATCCCATATGATGTGCTCCCT
GACTGGCTAAAGGACAACGACTATCTGCTACATGGTCATAGACCTCCCAT
GCCCTCCTTTCGGGCTTGCTTCAAGAGCATCTTCCGCATTCATACAGAAA
CTGGCAACATCTGGACCCATCTGCTTGGTTTCGTGCTGTTTCTCTTTTTG
GGAATCTTGACCATGCTCAGACCAAATATGTACTTCATGGCCCCTCTACA
GGAGAAGGTGGTTTTTGGGATGTTCTTTTTGGGTGCAGTGCTCTGCCTCA
GCTTCTCCTGGCTCTTTCACACCGTCTATTGTCATTCAGAGAAAGTCTCT
CGGACTTTTTCCAAACTGGACTATTCAGGGATTGCTCTTCTAATTATGGG
GAGCTTTGTCCCCTGGCTCTATTATTCCTTCTACTGCTCCCCACAGCCAC
GGCTCATCTACCTCTCCATCGTCTGTGTCCTGGGCATTTCTGCCATCATT
GTGGCGCAGTGGGACCGGTTTGCCACTCCTAAGCACCGGCAGACAAGAGC
AGGCGTGTTCCTGGGACTTGGCTTGAGTGGCGTCGTGCCCACCATGCACT
TTACTATCGCTGAGGGCTTTGTCAAGGCCACCACAGTGGGCCAGATGGGC
TGGTTCTTCCTCATGGCTGTGATGTACATCACTGGAGCTGGCCTTTATGC
TGCTCGAATTCCTGAGCGCTTCTTTCCTGGAAAATTTGACATATGGTTCC
AGTCTCATCAGATTTTCCATGTCCTGGTGGTGGCAGCAGCCTTTGTCCAC
TTCTATGGAGTCTCCAACCTTCAGGAATTCCGTTACGGCCTAGAAGGCGG
CTGTACTGATGACACCCTTCTC

The nucleotide sequence of EX-W01610-Lv03 Homo sapiens membrane frizzled-related protein (MFRP) (SEQ ID 10) is:

AACCCAGCTTTCTTGTACAAAGTGGTTGATCGCGTGCATGCGACGTCATAGCTCTCTCCCAA
TTCTCGACCTCGAGACAAATGGCAGTATTCATCCACAATTTTAAAAGAAAAGGGGGGATTGG
GGGGTACAGTGCAGGGGAAAGAATAGTAGACATAATAGCAACAGACATACAAACTAAAGAAT
TACAAAAACAAATTACAAAAATTCAAAATTTTCGGGTTTATTACAGGGACAGCAGAGATCCA
CTTTGGCCGCGGCTCGAGGGGGTTGGGGTTGCGCCTTTTCCAAGGCAGCCCTGGGTTTGCGC
AGGGACGCGGCTGCTCTGGGCGTGGTTCCGGGAAACGCAGCGGCGCCGACCCTGGGTCTCGC
ACATTCTTCACGTCCGTTCGCAGCGTCACCCGGATCTTCGCCGCTACCCTTGTGGGCCCCCC
GGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCA
ATGGCAGCGCGCCGACCGCGATGGGCTGTGGCCAATAGCGGCTGCTCAGCAGGGCGCGCCGA
GAGCAGCGGCCGGGAAGGGGCGGTGCGGGAGGCGGGGTGTGGGGCGGTAGTGTGGGCCCTGT
TCCTGCCCGCGCGGTGTTCCGCATTCTGCAAGCCTCCGGAGCGCACGTCGGCAGTCGGCTCC
CTCGTTGACCGAATCACCGACCTCTCTCCCCAGGGGGATCCACCGGAGCTTACCATGACCGA
GTACAAGCCCACGGTGCGCCTCGCCACCCGCGACGACGTCCCCAGGGCGGTACGCACCCTCG
CCGCCGCGTTCGCCGACTACCCCGCCACGCGCCACACCGTCGATCCGGACCGCCACATCGAG
CGGGTCACCGAGCTGCAAGAACTCTTCCTCACGCGCGTCGGGCTCGACATCGGCAAGGTGTG
GGTCGCGGACGACGGCGCCGCGGTGGCGGTCTGGACCACGCCGGAGAGCGTCGAAGCGGGGG
CGGTGTTCGCCGAGATCGGCCCGCGCATGGCCGAGTTGAGCGGTTCCCGGCTGGCCGCGCAG
CAACAGATGGAAGGCCTCCTGGCGCCGCACCGGCCCAAGGAGCCCGCGTGGTTCCTGGCCAC
CGTCGGCGTCTCGCCCGACCACCAGGGCAAGGGTCTGGGCAGCGCCGTCGTGCTCCCCGGAG
TGGAGGCGGCCGAGCGCGCCGGGGTGCCCGCCTTCCTGGAGACCTCCGCGCCCCGCAACCTC
CCCTTCTACGAGCGGCTCGGCTTCACCGTCACCGCCGACGTCGAGGTGCCCGAAGGACCGCG
CACCTGGTGCATGACCCGCAAGCCCGGTGCCTGACGCCCGCCCCACGACCCGCAGCGCCCGA
CCGAAAGGAGCGCACGACCCCATGCATCGGTACCTTTAAGACCAATGACTTACAAGGCAGCT
GTAGATCTTAGCCACTTTTTAAAAGAAAAGGGGGGACTGGAAGGGCTAATTCACTCCCAACG
AAGACAAGATCTGCTTTTTGCTTGTACTGGGTCTCTCTGGTTAGACCAGATCTGAGCCTGGG
AGCTCTCTGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGACCCTTTTA
GTCAGTGTGGAAAATCTCTAGCAGTAGTAGTTCATGTCATCTTATTATTCAGTATTTATAAC
TTGCAAAGAAATGAATATCAGAGAGTGAGAGGAACTTGTTTATTGCAGCTTATAATGGTTAC
AAATAAAGCAATAGCATCACAAATTTCACAAATAAAGCATTTTTTTCACTGCATTCTAGTTG
TGGTTTGTCCAAACTCATCAATGTATCTTATCATGTCTGGCTCTAGCTATCCCGCCCCTAAC
TCCGCCCATCCCGCCCCTAACTCCGCCCAGTTCCGCCCATTCTCCGCCCCATGGCTGACTAA
TTTTTTTTATTTATGCAGAGGCCGAGGCCGCCTCGGCCTCTGAGCTATTCCAGAAGTAGTGA
GGAGGCTTTTTTGGAGGCCTAGGGACGTACCCAATTCGCCCTATAGTGAGTCGTATTACGCG
CGCTCACTGGCCGTCGTTTTACAACGTCGTGACTGGGAAAACCCTGGCGTTACCCAACTTAA
TCGCCTTGCAGCACATCCCCCTTTCGCCAGCTGGCGTAATAGCGAAGAGGCCCGCACCGATC
GCCCTTCCCAACAGTTGCGCAGCCTGAATGGCGAATGGGACGCGCCCTGTAGCGGCGCATTA
AGCGCGGCGGGTGTGGTGGTTACGCGCAGCGTGACCGCTACACTTGCCAGCGCCCTAGCGCC
CGCTCCTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTCGCCCTTT
GACGTTGGAGTCCACGTTCTTTAATAGTGGACTCTTGTTCCAAACTGGAACAACACTCAACC
CTATCTCGGTCTATTCTTTTGATTTATAAGGGATTTTGCCGATTTCGGCCTATTGGTTAAAA
AATGAGCTGATTTAACAAAAATTTAACGCGAATTTTAACAAAATATTAACGCTTACAATTTA
GGTGGCACTTTTCGGGGAAATGTGCGCGGAACCCCTATTTGTTTATTTTTCTAAATACATTC
AAATATGTATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTTTGCCTT
CCTGTTTTTGCTCACCCAGAAACGCTGGTGAAAGTAAAAGATGCTGAAGATCAGTTGGGTGC
ACGAGTGGGTTACATCGAACTGGATCTCAACAGCGGTAAGATCCTTGAGAGTTTTCGCCCCG
AAGAACGTTTTCCAATGATGAGCACTTTTAAAGTTCTGCTATGTGGCGCGGTATTATCCCGT
ATTGACGCCGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATTATGCAGTG
CTGCCATAACCATGAGTGATAACACTGCGGCCAACTTACTTCTGACAACGATCGGAGGACCG
AAGGAGCTAACCGCTTTTTTGCACAACATGGGGGATCATGTAACTCGCCTTGATCGTTGGGA
ACCGGAGCTGAATGAAGCCATACCAAACGACGAGCGTGACACCACGATGCCTGTAGCAATGG
CAACAACGTTGCGCAAACTATTAACTGGCGAACTACTTACTCTAGCTTCCCGGCAACAATTA
ATAGACTGGATGGAGGCGGATAAAGTTGCAGGACCACTTCTGCGCTCGGCCCTTCCGGCTGG
CTGGTTTATTGCTGATAAATCTGGAGCCGGTGAGCGTGGGTCTCGCGGTATCATTGCAGCAC
TGGGGCCAGATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCATTGGTAACT
GTCAGACCAAGTTTACTCATATATACTTTAGATTGATTTAAAACTTCATTTTTAATTTAAAA
GGATCTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCAAAT
ACTGTTCTTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGA
GCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGGGGGGCG
GAGCCTATGGAAAAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGTATTACCGCCTTT
GAGTGAGCTGATACCGCTCGCCGCAGCCGAACGACCGAGCGCAGCGAGTCAGTGAGCGAGGA
AGCGGAAGAGCGCCCTTTGAGTGAGCTGATACCGCTCGCCGCAGCCGAACGACCGAGCGCAG
CGAGTCAGTGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCAGTGAGCGC
AACGCAAAATGTGAGTTAGCTCACTCATTAGGCACCCCAGGCTTTACACTTTATGCTTCCGG
CTCGTATGTTGTGTGGAATTGTGAGCGGATAACAATTTCACACAGGAAACAGCTATGACCAT
GATTACGCCAAGCGCGCAATTAACCCTCACTAAAGGGAACAAAAGCTGGAGCTGCAAGCTTA
ATGTAGTCTTATGCAATACTCTTGTAGTCTTGCAACATGGTAACGATGAGTTAGCAACATGC
CTTACAAGGAGAGAAAAAGCACCGTGCATGCCGATTGGTGGAAGTAAGGTGGTACGATCGTG
CCTTATTAGGAAGGCAACAGACGGGTCTGACATGGATTGGACGAACCACTGAATTGCCGCAT
TGCAGAGATATTGTATTTAAGTGCCTAGCTCGATACATAAACGGGTCTCTCTGGTTAGACCA
GATCTGAGCCTGGGAGCTCTCTGGCTAACTAGGGAACCCACTGCTTAAGCCTCAATAAAGCT
TGCCTTGAGTGCTTCAAGTAGTGTGTGCCCGTCTGTTGTGTGACTCTGGTAACTAGAGATCC
CTCAGACCCTTTTAGTCAGTGTGGAAAATCTCTAGCAGTGGCGCCCGAACAGGGACTTGAAA
GCGAAAGGGAAACCAGAGGAGCTCTCTCGACGCAGGACTCGGCTTGCTGAAGCGCGCACGGC
AAGAGGCGAGGGGCGGCGACTGGTGAGTACGCCAAAAATTTTGACTAGCGGAGGCTAGAAGG
AGAGAGATGGGTGCGAGAGCGTCAGTATTAAGCGGGGGAGAATTAGATCGCGATGGGAAAAA
ATTCGGTTAAGGCCAGGGGGAAAGAAAAAATATAAATTAAAACATATAGTATGGGCAAGCAG
GGAGCTAGAACGATTCGCAGTTAATCCTGGCCTGTTAGAAACATCAGAAGGCTGTAGACAAA
TACTGGGACAGCTACAACCATCCCTTCAGACAGGATCAGAAGAACTTAGATCATTATATAAT
ACAGTAGCAACCCTCTATTGTGTGCATCAAAGGATAGAGATAAAAGACACCAAGGAAGCTTT
AGACAAGATAGAGGAAGAGCAAAACAAAAGTAAGACCACCGCACAGCAAGCGGCCGCTGATC
TTCAGACCTGGAGGAGGAGATATGAGGGACAATTGGAGAAGTGAATTATATAAATATAAAGT
AGTAAAAATTGAACCATTAGGAGTAGCACCCACCAAGGCAAAGAGAAGAGTGGTGCAGAGAG
AAAAAAGAGCAGTGGGAATAGGAGCTTTGTTCCTTGGGTTCTTGGGAGCAGCAGGAAGCACT
ATGGGCGCAGCGTCAATGACGCTGACGGTACAGGCCAGACAATTATTGTCTGGTATAGTGCA
GCAGCAGAACAATTTGCTGAGGGCTATTGAGGCGCAACAGCATCTGTTGCAACTCACAGTCT
GGGGCATCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGGAATGC
TAGTTGGAGTAATAAATCTCTGGAACAGATTTGGAATCACACGACCTGGATGGAGTGGGACA
GAGAAATTAACAATTACACAAGCTTAATACACTCCTTAATTGAAGAATCGCAAAACCAGCAA
GAAAAGAATGAACAAGAATTATTGGAATTAGATAAATGGGCAAGTTTGTGGAATTGGTTTAA
CATAACAAATTGGCTGTGGTATATAAAATTATTCATAATGATAGTAGGAGGCTTGGTAGGTT
TAAGAATAGTTTTTGCTGTACTTTCTATAGTGAATAGAGTTAGGCAGGGATATTCACCATTA
TCGTTTCAGACCCACCTCCCAACCCCGAGGGGACCCGACAGGCCCGAAGGAATAGAAGAAGA
AGGTGGAGAGAGAGACAGAGACAGATCCATTCGATTAGTGAACGGATCTCGACGGTATCGAT
CACGAGACTAGCCTCGAGCGGCCGCCCCCTTCACCGAGGGCCTATTTCCCATGATTCCTTCA
TATTTGCATATACGATACAAGGCTGTTAGAGAGATAATTGGAATTAATTTGACTGTAAACAC
AAAGATATTAGTACAAAATACGTGACGTAGAAAGTAATAATTTCTTGGGTAGTTTGCAGTTT
TAAAATTATGTTTTAAAATGGACTATCATATGCTTACCGTAACTTGAAAGTATTTCGATTTC
TTGGCTTTATATATCTTGTGGAAAGGACGAAACACCGGTATCTATACATTGAATCAATATTG
GCAATTAGCCATATTAGTCATTGGTTATATAGCATAAATCAATATTGGCTATTGGCCATTGC
ATACGTTGTATCTATATCATAATATGTACATTTATATTGGCTCATGTCCAATATGACCGCCA
TGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTACATCAAGT
GTATCATATGCCAAGTCCGCCCCCTATTGACGTCAATGACGGTAAATGGCCCGCCTGGCATT
ATGCCCAGTACATGACCTTACGGGACTTTCCTACTTGGCAGTACATCTACGTATTAGTCATC
GCTATTACCATGGTGATGCGGTTTTGGCAGTACATCAATGGGCGTGGATAGCGGTTTGACTC
ACGGGGATTTCCAAGTCTCCACCCCATTGACGTCAATGGGAGTTTGTTTTGGCACCAAAATC
AACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGTCAGATCGCCTGGAGACG
CCATCCACGCTGTTTTGACCTCCATAGAAGATTCTAGAACCATGGTGAGCAAGGGCGAGGAG
CTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCT
GCACCACCGGCAAGCTGCCCGTGCCCTGGCCCACCCTCGTGACCACCCTGACCTACGGCGTG
CAGTGCTTCAGCCGCTACCCCGACCACATGAAGCAGCACGACTTCTTCAAGTCCGCCATGCC
CGAAGGCTACGTCCAGGAGCGCACCATCTTCTTCAAGGACGACGGCAACTACAAGACCCGCG
CCGAGGTGAAGTTCGAGGGCGACACCCTGGTGAACCGCATCGAGCTGAAGGGCATCGACTTC
AAGGAGGACGGCAACATCCTGGGGCACAAGCTGGAGTACAACTACAACAGCCACAACGTCTA
TATCATGGCCGACAAGCAGAAGAACGGCATCAAGGTGAACTTCAAGATCCGCCACAACATCG
AGGACGGCAGCGTGCAGCTCGCCGACCACTACCAGCAGAACACCCCCATCGGCGACGGCCCC
GTGCTGCTGCCCGACAACCACTACCTGAGCACCCAGTCCGCCCTGAGCAAAGACCCCAACGA
GAAGCGCGATCACATGGTCCTGCTGGAGTTCGTGACCGCCGCCGGGATCACTCTCGGCATGG

[chemical structure]

[chemical structure]

The nucleotide sequence of EX-Z1681-Lv111 Homo sapiens adiponectin receptor 1 (ADIPOR1) (SEQ ID 11) is:

AACCCAGCTTTCTTGTACAAAGTGGTTGATCGCGTGCATGCGACGTCATAGCTCTCTCCC
AATTCTCGACCTCGAGACAAATGGCAGTATTCATCCACAATTTTAAAAGAAAAGGGGGGA
TTGGGGGGTACAGTGCAGGGGAAAGAATAGTAGACATAATAGCAACAGACATACAAACTA
AAGAATTACAAAAACAAATTACAAAAATTCAAAATTTTCGGGTTTATTACAGGGACAGCA
GAGATCCACTTTGGCCGCGGCTCGAGGGGGTTGGGGTTGCGCCTTTTCCAAGGCAGCCCT
GGGTTTGCGCAGGGACGCGGCTGCTCTGGGCGTGGTTCCGGGAAACGCAGCGGCGCCGAC
CCTGGGTCTCGCACATTCTTCACGTCCGTTCGCAGCGTCACCCGGATCTTCGCCGCTACC
CTTGTGGGCCCCCCGGCGACGCTTCCTGCTCCGCCCCTAAGTCGGGAAGGTTCCTTGCGG
TTCGCGGCGTGCCGGACGTGACAAACGGAAGCCGCACGTCTCACTAGTACCCTCGCAGAC
GGACAGCGCCAGGGAGCAATGGCAGCGCGCCGACCGCGATGGGCTGTGGCCAATAGCGGC
TGCTCAGCAGGGCGCGCCGAGAGCAGCGGCCGGGAAGGGGCGGTGCGGGAGGCGGGGTGT
GGGGCGGTAGTGTGGGCCCTGTTCCTGCCCGCGCGGTGTTCCGCATTCTGCAAGCCTCCG
GAGCGCACGTCGGCAGTCGGCTCCCTCGTTGACCGAATCACCGACCTCTCTCCCCAGGGG
GATCCACCGGAGCTTACCATGACCGAGTACAAGCCCACGGTGCGCCTCGCCACCCGCGAC
GACGTCCCCAGGGCGGTACGCACCCTCGCCGCCGCGTTCGCCGACTACCCCGCCACGCGC
CACACCGTCGATCCGGACCGCCACATCGAGCGGGTCACCGAGCTGCAAGAACTCTTCCTC
ACGCGCGTCGGGCTCGACATCGGCAAGGTGTGGGTCGCGGACGACGGCGCCGCGGTGGCG
GTCTGGACCACGCCGGAGAGCGTCGAAGCGGGGGCGGTGTTCGCCGAGATCGGCCCGCGC
ATGGCCGAGTTGAGCGGTTCCCGGCTGGCCGCGCAGCAACAGATGGAAGGCCTCCTGGCG
CCGCACCGGCCCAAGGAGCCCGCGTGGTTCCTGGCCACCGTCGGCGTCTCGCCCGACCAC
CAGGGCAAGGGTCTGGGCAGCGCCGTCGTGCTCCCCGGAGTGGAGGCGGCCGAGCGCGCC
GGGGTGCCCGCCTTCCTGGAGACCTCCGCGCCCCGCAACCTCCCCTTCTACGAGCGGCTC
GGCTTCACCGTCACCGCCGACGTCGAGGTGCCCGAAGGACCGCGCACCTGGTGCATGACC
CGCAAGCCCGGTGCCTGACGCCCGCCCCACGACCCGCAGCGCCCGACCGAAAGGAGCGCA
CGACCCCATGCATCGGTACCTTTAAGACCAATGACTTACAAGGCAGCTGTAGATCTTAGC
CACTTTTTAAAAGAAAAGGGGGGACTGGAAGGGCTAATTCACTCCCAACGAAGACAAGAT
CTGCTTTTTGCTTGTACTGGGTCTCTCTGGTTAGACCAGATCTGAGCCTGGGAGCTCTCT
GGCTAACTAGGGAACCCACTGCTTAAGCCTCAATAAAGCTTGCCTTGAGTGCTTCAAGTA
GTGTGTGCCCGTCTGTTGTGTGACTCTGGTAACTAGAGATCCCTCAGACCCTTTTAGTCA
GTGTGGAAAATCTCTAGCAGTAGTAGTTCATGTCATCTTATTATTCAGTATTTATAACTT
GCAAAGAAATGAATATCAGAGAGTGAGAGGAACTTGTTTATTGCAGCTTATAATGGTTAC
AAATAAAGCAATAGCATCACAAATTTCACAAATAAAGCATTTTTTTCACTGCATTCTAGT
TGTGGTTTGTCCAAACTCATCAATGTATCTTATCATGTCTGGCTCTAGCTATCCCGCCCC
TAACTCCGCCCATCCCGCCCCTAACTCCGCCCAGTTCCGCCCATTCTCCGCCCCATGGCT
GACTAATTTTTTTTATTTATGCAGAGGCCGAGGCCGCCTCGGCCTCTGAGCTATTCCAGA
AGTAGTGAGGAGGCTTTTTTGGAGGCCTAGGGACGTACCCAATTCGCCCTATAGTGAGTC
GTATTACGCGCGCTCACTGGCCGTCGTTTTACAACGTCGTGACTGGGAAAACCCTGGCGT
TACCCAACTTAATCGCCTTGCAGCACATCCCCCTTTCGCCAGCTGGCGTAATAGCGAAGA
GGCCCGCACCGATCGCCCTTCCCAACAGTTGCGCAGCCTGAATGGCGAATGGGACGCGCC
CTGTAGCGGCGCATTAAGCGCGGCGGGTGTGGTGGTTACGCGCAGCGTGACCGCTACACT
TGCCAGCGCCCTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTGGGCCATCGCC
CTGATAGACGGTTTTTCGCCCTTTGACGTTGGAGTCCACGTTCTTTAATAGTGGACTCTT
GTTCCAAACTGGAACAACACTCAACCCTATCTCGGTCTATTCTTTTGATTTATAAGGGAT
TTTGCCGATTTCGGCCTATTGGTTAAAAAATGAGCTGATTTAACAAAAATTTAACGCGAA
TTTTAACAAAATATTAACGCTTACAATTTAGGTGGCACTTTTCGGGGAAATGTGCGCGGA
ACCCCTATTTGTTTATTTTTCTAAATACATTCAAATATGTATCCGCTCATGAGACAATAA
CCCTGATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAATGATG
AGCACTTTTAAAGTTCTGCTATGTGGCGCGGTATTATCCCGTATTGACGCCGGGCAAGAG
CAACTCGGTCGCCGCATACACTATTCTCAGAATGACTTGGTTGAGTACTCACCAGTCACA
GAAAAGCATCTTACGGATGGCATGACAGTAAGAGAATTATGCAGTGCTGCCATAACCATG
AGTGATAACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGGAGCTG
AATGAAGCCATACCAAACGACGAGCGTGACACCACGATGCCTGTAGCAATGGCAACAACG
TTGCGCAAACTATTAACTGGCGAACTACTTACTCTAGCTTCCCGGCAACAATTAATAGAC
TGGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACTG
GGGCCAGATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCATTGGTAA
CTGTCAGACCAAGTTTACTCATATATACTTTAGATTGATTTAAAACTTCATTTTTAATTT
AAAAGGATCTAGGTGAAGATCCTTTTTGATAATCTCATGACCAAAATCCCTTAACGTGAG
TTTTCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGAGCG
CAGATACCAAATACTGTTCTTCTAGTGTAGCCGTAGTTAGGCCACCACTTCAAGAACTCT
GTAGCACCGCCTACATACCTCGCTCTGCTAATCCTGTTACCAGTGGCTGCTGCCAGTGGC
GATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAA
CTGAGATACCTACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGAGCTTCCAGGG
GGAAACGCCTGGTATCTTTATAGTCCTGTCGGGTTTCGCCACCTCTGACTTGAGCGTCGA
TTTTTGTGATGCTCGTCAGGGGGGCGGAGCCTATGGAAAAACGCCAGCAACGCGGCCTTT
TTACGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAGAGCGCCCTTTGAGTGAGCTG
ATACCGCTCGCCGCAGCCGAACGACCGAGCGCAGCGAGTCAGTGAGCGAGGAAGCGGAAG
AGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAAAATGTGAGTTAGCTC
ACTCATTAGGCACCCCAGGCTTTACACTTTATGCTTCCGGCTCGTATGTTGTGTGGAATT
GTGAGCGGATAACAATTTCACACAGGAAACAGCTATGACCATGATTACGCCAAGCGCGCA
ATTAACCCTCACTAAAGGGAACAAAAGCTGGAGCTGCAAGCTTAATGTAGTCTTATGCAA
TACTCTTGTAGTCTTGCAACATGGTAACGATGAGTTAGCAACATGCCTTACAAGGAGAGA
AAAAGCACCGTGCATGCCGATTGGTGGAAGTAAGGTGGTACGATCGTGCCTTATTAGGAA
GGCAACAGACGGGTCTGACATGGATTGGACGAACCACTGAATTGCCGCATTGCAGAGATA
TTGTATTTAAGTGCCTAGCTCGATACATAAACGGGTCTCTCTGGTTAGACCAGATCTGAG
CCTGGGAGCTCTCTGGCTAACTAGGGAACCCACTGCTTAAGCCTCAATAAAGCTTGCCTT
GAGTGCTTCAAGTAGTGTGTGCCCGTCTGTTGTGTGACTCTGGTAACTAGAGATCCCTCA
GACCCTTTTAGTCAGTGTGGAAAATCTCTAGCAGTGGCGCCCGAACAGGGACTTGAAAGC
GAAAGGGAAACCAGAGGAGCTCTCTCGACGCAGGACTCGGCTTGCTGAAGCGCGCACGGC
AAGAGGCGAGGGGCGGCGACTGGTGAGTACGCCAAAAATTTTGACTAGCGGAGGCTAGAA
GGAGAGAGATGGGTGCGAGAGCGTCAGTATTAAGCGGGGGAGAATTAGATCGCGATGGGA
AAAAATTCGGTTAAGGCCAGGGGGAAAGAAAAAATATAAATTAAAACATATAGTATGGGC
AAGCAGGGAGCTAGAACGATTCGCAGTTAATCCTGGCCTGTTAGAAACATCAGAAGGCTG
TAGACAAATACTGGGACAGCTACAACCATCCCTTCAGACAGGATCAGAAGAACTTAGATC
ATTATATAATACAGTAGCAACCCTCTATTGTGTGCATCAAAGGATAGAGATAAAAGACAC
CAAGGAAGCTTTAGACAAGATAGAGGAAGAGCAAAACAAAAGTAAGACCACCGCACAGCA
AGCGGCCGCTGATCTTCAGACCTGGAGGAGGAGATATGAGGGACAATTGGAGAAGTGAAT
TATATAAATATAAAGTAGTAAAAATTGAACCATTAGGAGTAGCACCCACCAAGGCAAAGA
GAAGAGTGGTGCAGAGAGAAAAAAGAGCAGTGGGAATAGGAGCTTTGTTCCTTGGGTTCT
TGGGAGCAGCAGGAAGCACTATGGGCGCAGCGTCAATGACGCTGACGGTACAGGCCAGAC
AATTATTGTCTGGTATAGTGCAGCAGCAGAACAATTTGCTGAGGGCTATTGAGGCGCAAC
AGCATCTGTTGCAACTCACAGTCTGGGGCATCAAGCAGCTCCAGGCAAGAATCCTGGCTG
TGGAAAGATACCTAAAGGATCAACAGCTCCTGGGGATTTGGGGTTGCTCTGGAAAACTCA
TTTGCACCACTGCTGTGCCTTGGAATGCTAGTTGGAGTAATAAATCTCTGGAACAGATTT
GGAATCACACGACCTGGATGGAGTGGGACAGAGAAATTAACAATTACACAAGCTTAATAC
ACTCCTTAATTGAAGAATCGCAAAACCAGCAAGAAAAGAATGAACAAGAATTATTGGAAT
TAGATAAATGGGCAAGTTTGTGGAATTGGTTTAACATAACAAATTGGCTGTGGTATATAA
AATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAA
CCCCGAGGGGACCCGACAGGCCCGAAGGAATAGAAGAAGAAGGTGGAGAGAGAGACAGAG
ACAGATCCATTCGATTAGTGAACGGATCTCGACGGTATCGATCACGAGACTAGCCTCGAG
CGGCCGCCCCCTTCACCGAGGGCCTATTTCCCATGATTCCTTCATATTTGCATATACGAT
ACAAGGCTGTTAGAGAGATAATTGGAATTAATTTGACTGTAAACACAAAGATATTAGTAC
AAAATACGTGACGTAGAAAGTAATAATTTCTTGGGTAGTTTGCAGTTTTAAAATTATGTT
TTAAAATGGACTATCATATGCTTACCGTAACTTGAAAGTATTTCGATTTCTTGGCTTTAT
ATATCTTGTGGAAAGGACGAAACACCGGTATCTATACATTGAATCAATATTGGCAATTAG
CCATATTAGTCATTGGTTATATAGCATAAATCAATATTGGCTATTGGCCATTGCATACGT
TGTATCTATATCATAATATGTACATTTATATTGGCTCATGTCCAATATGACCGCCATGTT
GACATTGATTATTGACTAGTTATTAATAGTAATCAATTACGGGGTCATTAGTTCATAGCC
CATATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGTACATC
AAGTGTATCATATGCCAAGTCCGCCCCCTATTGACGTCAATGACGGTAAATGGCCCGCCT
GGCATTATGCCCAGTACATGACCTTACGGGACTTTCCTACTTGGCAGTACATCTACGTAT
TAGTCATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTTGTTTT
GGCACCAAAATCAACGGGACTTTCCAAAATGTCGTAACAACTCCGCCCCATTGACGCAAA
TGGGCGGTAGGCGTGTACGGTGGGAGGTCTATATAAGCAGAGCTCGTTTAGTGAACCGTC
AGATCGCCTGGAGACGCCATCCACGCTGTTTTGACCTCCATAGAAGATTCTAGAACCATG
GTGAGCAAGGGCGAGGAGGATAACATGGCCATCATCAAGGAGTTCATGCGCTTCAAGGTG
CACATGGAGGGCTCCGTGAACGGCCACGAGTTCGAGATCGAGGGCGAGGGCGAGGGCCGC
CCCTACGAGGGCACCCAGACCGCCAAGCTGAAGGTGACCAAGGGTGGCCCCCTGCCCTTC
GCCTGGGACATCCTGTCCCCTCAGTTCATGTACGGCTCCAAGGCCTACGTGAAGCACCCC
GCCGACATCCCCGACTACTTGAAGCTGTCCTTCCCCGAGGGCTTCAAGTGGGAGCGCGTG
ATGAACTTCGAGGACGGCGGCGTGGTGACCGTGACCCAGGACTCCTCCCTGCAGGACGGC
GAGTTCATCTACAAGGTGAAGCTGCGCGGCACCAACTTCCCCTCCGACGGCCCCGTAATG
CAGAAGAAGACCATGGGCTGGGAGGCCTCCTCCGAGCGGATGTACCCCGAGGACGGCGCC
CTGAAGGGCGAGATCAAGCAGAGGCTGAAGCTGAAGGACGGCGGCCACTACGACGCTGAG
GTCAAGACCACCTACAAGGCCAAGAAGCCCGTGCAGCTGCCCGGCGCCTACAACGTCAAC
ATCAAGTTGGACATCACCTCCCACAACGAGGACTACACCATCGTGGAACAGTACGAACGC

[chemical structure]

[chemical structure]

[chemical structure]

[chemical structure]

In embodiments, the genetically engineered cell expresses a transgene encoding AdipoR1, MFRP, or both AdipoR1 and MFRP. In embodiments, the transgene comprises AdipoR1 human variant 4 (Accession No. NM_001290553), human MFRP (Accession No. NM_031433), and/or MFRP mouse variant 2 (Accession No. NM_001190314). In still other embodiments, the genetically engineered cell expresses one or more transgenes encoding membrane-bound proteins implicated in omega-3 fatty acid uptake and retention, such as those proteins involved in the transport of omega-3 fatty acids across the cellular membrane.

AdipoR1 is a cell-surface receptor that mediates docosahexaenoic acid (DHA) cellular uptake and retention, a process needed for the production of the neuroprotective lipids, such as docosanoids and elavanoids, and other bioactive compound which can be cytoprotective. For example, after AdipoR1-mediated uptake of DHA into the cell, and DHA metabolism will result in the formation of bioactive and/or cytoprotective compounds, such as the neuroprotective docosanoid NPD1.

MFRP has a similar function. Notably, MFRP captures DHA into photoreceptor cells. The deletion of MFRP causes retinal degenerations (photoreceptor cell degenerations).

In embodiments, the transgene can comprise synthetic polynucleotide, which can refer to a polynucleotide sequence that does not exist in nature but instead is made by the hand of man, either chemically, or biologically (i.e., in vitro modified). For example, the synthetic polynucleotide can be made using cloning and vector propagation techniques.

The term “vector” can refer to nucleic acid molecules, usually double-stranded DNA, which may have inserted into it, such as within its backbone or coding region, another nucleic acid molecule (the insert nucleic acid molecule) such as, but not limited to, a cDNA molecule. The vector can be used to transport the insert nucleic acid molecule into a suitable host cell. A vector can contain the elements necessary to permit transcribing the insert nucleic acid molecule, and, optionally, translating the transcript into a polypeptide. The insert nucleic acid molecule can be derived from the host cell, or may be derived from a different cell or organism. Once in the host cell, the vector can replicate independently of, or coincidental with, the host chromosomal DNA, and several copies of the vector and its inserted nucleic acid molecule may be generated (Sambrook et al., Molecular Cloning: A Laboratory Manual, Cold SpringHarbor Laboratory, Cold Spring Harbor, 1989).

Further, vector can also refer to a gene delivery vehicle that facilitates gene transfer into a target cell and can include both non-viral and viral vectors. Non-viral vectors include but are not limited to cationic lipids, liposomes, nanoparticles, PEG, and PEI. Viral vectors are derived from viruses and include but are not limited to retrovirus, lentivirus, adeno-associated virus, adenovirus, herpesvirus, and hepatitis virus. Viral vectors can be replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis.

In embodiments, vectors can be derived from adeno-associated virus, adenovirus, retroviruses and Antiviruses. Alternatively, gene delivery systems can be used to combine viral and non-viral components, such as nanoparticles or virosomes (Yamada, Tadanori, et al. “Nanoparticles for the delivery of genes and drugs to human hepatocytes.” Nature biotechnology 21.8 (2003): 885-890). Retroviruses and Antiviruses are RNA viruses that have the ability to insert their genes into host cell chromosomes after infection. Retroviral and lentiviral vectors have been developed that lack the genes encoding viral proteins, but retain the ability to infect cells and insert their genes into the chromosomes of the target cell (Miller, Daniel G., Mohammed A. Adam, and A. Dusty Miller. “Gene transfer by retrovirus vectors occurs only in cells that are actively replicating at the time of infection.” Molecular and cellular biology 10.8 (1990): 4239-4242; Naldini, Luigi, et al. “In vivo gene delivery and stable transduction of nondividing cells by a lentiviral vector.” Science 272.5259 (1996): 263, VandenDriessche, Thierry, et al. “Long-term expression of human coagulation factor VIII and correction of hemophilia A after in vivo retroviral gene transfer in factor VIII-deficient mice.” Proceedings of the National Academy of Sciences 96.18 (1999): 10379-10384). The difference between a lentiviral and a classical Moloney-murine leukemia-virus (MLV) based retroviral vector is that lentiviral vectors can transduce both dividing and non-dividing cells whereas MLV-based retroviral vectors can only transduce dividing cells.

The genetically engineered cell of the claimed invention can express transgenes as described herein from vectors, non-limiting examples of which comprise viral vectors, plasmids, cosmids, and artificial chromosomes.

A “plasmid,” for example a bacterial plasmid, can refer to a DNA molecule with a cell that is physically separated from a chromosomal DNA and can replicate independently.

A “cosmid” can refer to a plasmid vector that contains a cos sequence.

An “artificial chromosome” can refer to a nucleic acid sequence of a chromosome that is constructed from a series of smaller nucleic acid sequences. For example, the smaller sequences are constructed into bacterial artificial chromosomes (BACS) or yeast artificial chromosomes (YACS).

A “viral vector” can refer to a virus that is competent to infect a mammalian host cell and/or can be used to deliver a construct to a target cells or to an animal systemically. One example of a viral vector is the first generation E1/E3 deleted nonreplicating Ad5 vector, but other forms of viral delivery systems are known and could be used. One of the disadvantages of the non-replicating adenovirus is the lack of persistence in vivo and one embodiment could be the use of a conditionally replicating oncolytic adenovirus. Additional examples of viral delivery systems comprise viruses that would result in more permanent expression such as lentivirus or adeno-associated virus (AAV). The advantage to these two viral systems is that they can be manipulated to alter their tropism for different cell types making them a more flexible platform.

There are several types of viral vectors that can be used to deliver nucleic acids into the genetic makeup of cells, non-limiting examples of which include retrovirus, lentivirus, adenovirus, adeno-associated virus and herpes simplex virus. For example, the vector can be a lentiviral vector, such as pReceiver.

Such vectors, also known as expression vectors or DNA expression constructs, can be modified to include and/or be operably linked to regulatory elements to carry out the embodiments of this invention. As used herein, the term “expression vector” can refer to a plasmid origin, a promoter and/or enhancer, one or more transgenes, a transcription terminator, and optionally a selection gene. Additionally, such vectors can contain multipurpose cloning regions that have numerous restriction enzyme sites.

Embodiments can contain markers for selection of cells that are positively transfected with the vector. Non-limiting examples of such selection markers include antibiotic resistant genes, such as those that result in resistance to puromycin or ampicillin, or fluorescent markers, such as mCherry or EGFP, or a combination of selections markers.

Omega-3 Fatty Acids

Omega-3 fatty acids are polyunsaturated fatty acids that have the final double bond in the hydrocarbon chain between the third and fourth carbon atoms from the methyl end of the molecule. Non-limiting examples of omega-3 fatty acids include, 5,8,11,14,17-eicosapentaenoic acid (EPA), 4,7,10,13,16,19-docosahexanoic acid (DHA), 7,10,13,16,19-docosapentanoic acid (DPA), and α-linolenic acid (ALA).

Omega-3 fatty acids are important for normal metabolism. Mammals are unable to synthesize omega-3 fatty acids, but can obtain the shorter-chain omega-3 fatty acid ALA (18 carbons and 3 double bonds) through diet and use it to form the more important long-chain omega-3 fatty acids, EPA (20 carbons and 5 double bonds) and then from EPA, the most crucial, DHA (22 carbons and 6 double bonds). The ability to make the longer-chain omega-3 fatty acids from ALA may be impaired in aging.

In embodiments as described herein of genetically engineered cells transformed with at least one polynucleotide encoding a polypeptide, wherein the expressed polypeptide transports an Omega-3 fatty acid and/or a long chain fatty acid across the cell membrane, the omega-3 fatty acid is selected from at least one of Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA), Clupanodonic acid, Docosahexaenoic acid (DHA), Tetracosapentaenoic acid, Tetracosahexaenoic acid (Nisinic acid), or their fatty acid precursors thereof. Embodiments as described herein comprise any combination of omega-3 fatty acids.

The Cells

The invention is directed towards a genetically engineered cell line comprising a plurality of cells transformed with at least one polynucleotide encoding a protein, polypeptide, or fragment thereof that transports an Omega-3 fatty acid across the cell membrane and/or retains the fatty acid within the cell.

The term “genetically-engineered cell” can refer to a cell into which a foreign (i.e., non-naturally occurring) nucleic acid, e.g., DNA, has been introduced. The foreign nucleic acid can be introduced by a variety of techniques, including, but not limited to, calcium-phosphate-mediated transfection, DEAE-mediated transfection, microinjection, retroviral transformation, protoplast fusion and lipofection. The genetically-engineered cell can express the foreign nucleic acid in either a transient or long-term manner. For example, transient expression occurs when foreign DNA does not stably integrate into the chromosomal DNA of the transfected cell. In contrast, long-term expression of foreign DNA occurs when the foreign DNA has been stably integrated into the chromosomal DNA of the transfected cell.

Embodiments as described herein comprise a genetically engineered cell line comprising a plurality of cells transformed with at least one polynucleotide encoding a polypeptide, wherein the expressed polypeptide transports an Omega-3 fatty acid across the cell membrane.

In order to generate the genetically engineered cell as described herein, at least one polynucleotide encoding the protein, polypeptide, or fragment thereof that transports an Omega-3 fatty acid is introduced into the plurality of cells.

The polynucleotide, which can comprise DNA, RNA, or a fragment thereof, can be introduce into a plurality of cells any cell type. The term “cell” can refer to cytoplasm bound by a membrane that contains DNA within. A cell can be either a prokaryotic or eukaryotic cell.

For example, the cell can be isolated from a tissue from a human subject, non-limiting examples of such tissues comprise skin, adipose tissue, bone marrow, blood, human brain cells, pericytes, macrophages, or retinal pigment epithelial cells.

Non-limiting examples of cell types comprise skin fibroblasts, adipose tissue stem cells, primary retinal pigment epithelial cells, human adult stem cells, transdifferentiated neuronal cells, pericytes, and macrophages.

Further, the plurality of cells can be a stem cell, such as a pluripotent stem cell or a totipotent stem cell. Exemplary but non-limiting established lines of human embryonic stem (ES) cells include lines which are listed in the NIH Human Embryonic Stem Cell Registry (http://stemcells.nih.gov/research/registry), and sub-lines thereof. Other exemplary established hES cell lines include those deposited at the UK Stem Cell Bank (http://www.ukstemcellbank.org.uk/), and sub-lines thereof.

As used herein, a “stem cell” can refer to a cell, such as a progenitor cell, further capable of self-renewal, which can under appropriate conditions proliferate without differentiation. Stem cells can also be cells capable of substantial unlimited self-renewal, wherein at least a portion of the stem cell's progeny substantially retains the unspecialized or relatively less specialized phenotype, the differentiation potential, and the proliferation capacity of the mother stem cell. Stem cells can also be cells which display limited self-renewal, wherein the capacity of the stem cell's progeny for further proliferation and/or differentiation is demonstrably reduced compared to the mother cell.

As used herein, “pluripotent” can refer to a stem cell capable of giving rise to cell types originating from all three germ layers of an organism (i.e., mesoderm, endoderm, and ectoderm), and potentially capable of giving rise to any and all cell types of an organism, although not able to grow into the whole organism.

A progenitor or stem cell can refer to a cell that can “give rise” to another, relatively more specialized cell when, for example, the progenitor or stem cell differentiates to become said other cell without previously undergoing cell division, or if said other cell is produced after one or more rounds of cell division and/or differentiation of the progenitor or stem cell. A “mammalian pluripotent stem cell” or “mPS” cell can refer to a pluripotent stem cell of mammalian origin. The term “mammal” can refer to any animal classified as such, non-limiting examples of which include humans, domestic and farm animals, zoo animals, sport animals, pet animals, companion animals and experimental animals, such as, for example, mice, rats, hamsters, rabbits, dogs, cats, guinea pigs, cattle, cows, sheep, horses, pigs and primates (e.g., monkeys and apes).

The plurality of cells can be the product of transdifferentiation, a process wherein a non-neuronal cell is differentiated into a neuron or a neuron-like cell (Krabbe, Christina, Jens Zimmer, and Morten Meyer. “Neural transdifferentiation of mesenchymal stem cells—a critical review.” Apmis 113.11-12 (2005): 831-844). For example, the cell can be a skin fibroblast that upon trasdifferentiation becomes neuronal-like.

In other embodiments, the plurality of cells can be populations of cells, and subpopulations thereof, such as those distinguished and isolated from a sample population. Further, the plurality of cells can be neurons, neural precursor cells, mature neuron-like cells and populations or subpopulations thereof, such as hippocampal cells.

Without wishing to be bound by theory, the plurality of cells can comprise any cells that have characteristics of mammalian cells (i.e. mouse or human cells), pluripotent cells (i.e. embryonic stem cells or embryonic germ cells).

Method of Generating the Cells

The invention also provides for methods of generating genetically-engineered cells as described herein. For example, an embodiment comprises the step of obtaining a plurality of cells, and introducing into the cells at least one polynucleotide encoding a polypeptide that transports across the cell membrane or retains within a cell an Omega-3 fatty.

Embodiments can further comprise detecting the presence of the expression vector or the polypeptide within the plurality of cells, for example by antibiotic resistance screens, immunohistochemistry (such as Western blot analysis) or FACS. Also, the biological functions of the polypeptides can be confirmed, such as detecting an enhanced ability to take up deuterium-labeled DHA, an enhanced survival to oxidative stress, or a combination thereof.

The cells, such as skin fibroblasts, adipose tissue stem cells, primary retinal pigment epithelial cells, into which the polynucleotide is to be introduced into can be obtained from sources such as the subjects themselves, donor subjects, or cell banks. For example, the cells can be harvested from a subject, as is the case with stem cells that can be used for autologous transplantation as described herein.

In embodiments, the polynucleotide can be introduced into the cells by transduction, such as transfer by bacteriophages or viruses; transformation, such as uptake of naked DNA from outside of the cell; or microinjection.

In an embodiment, high titer lentiviral stocks can be used for the generation of stable clones constitutively expressing at least one of the ADIPOR1 (and/or MFRP) protein, such as SEQ ID 3-7, along with plasmid constructs that contain a cassette for either mCherry (for ADIPOR1) or eGFP (for MFRP) and both contain a puromycin resistance cassette. In embodiments, the lentiviral stocks comprise SEQ ID 1 and/or SEQ ID 2.

As needed, positive and negative controls can be used. For example, positive controls for transduction efficiency can be empty plasmids lentiviral stocks carrying the mCherry, eGFP, or other fluorescent molecular tags, such as YFP, BFP, or RFP.

Steps to generate stable clones constitutively expressing at least one of the ADIPOR1 (and/or MFRP) proteins, such as SEQ ID 3-7, comprise seeding cells into onto a support medium, for example seeding ˜2×10⁴ cells in 6-well plates, and letting the cells adhere overnight. Next, replacing media with fresh complete media containing serial dilutions of 10, 5, 2.5, 1.25 and 0.125 μls of lentiviral stock, such as those that comprise SEQ ID 1 and/or SEQ ID 2, including one well with fresh media alone (negative control). Dedicating one plate for each expression plasmid (for example, ADIPOR1-mCherry, mCherry and eGFP). In parallel similar transduction will be done in optical-six well plates.

Day 1 after transduction, cell health/viability can assessed, such as by inspection. Transduction media will be discarded and replaced with fresh complete media containing 2 μg/ml puromycin. Media supplemented with puromycin will be changed every day until all cells are dead in the mock transduction well.

Day 3 after transduction, efficiency of transduction can be assessed, such as by confocal microscopy and western blot analysis. For example, the optical-bottom plates will be used for mCherry-red fluorescence measurements by confocal microscopy. Thereafter cells can be detached with trypsin-EDTA, processed for western blot analysis of ADIPOR1 expression using in house protocols and reagents. Day 10-20 after transduction, surviving clones can be pooled together, for example to assess “clonal effect.” Specific transgene expression can be assessed by western blot, qRT-PCR and sequencing.

The Composition

The invention provides for a therapeutic composition, or a “pharmaceutical composition” or “formulation” comprising a plurality of genetically engineered cells as described herein and a pharmaceutically acceptable carrier.

As used herein, a “pharmaceutical composition” or a “pharmaceutical formulation” can refer to a composition or pharmaceutical composition suitable for administration to a subject, such as a mammal, especially a human, and that refers to the combination of an active agent(s) (e.g., genetically engineered cell), or ingredient with a pharmaceutically acceptable carrier or excipient, making the composition suitable for diagnostic, therapeutic, or preventive use in vitro, in vivo, or ex vivo. According to the invention, a pharmaceutical composition can be sterile and free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

Further, the pharmaceutical composition can contain components that ensure the viability of the cells therein. In particular, the cells can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996, which is incorporated herein in its entirety. Choice of the cellular excipient and any accompanying elements of the composition will be adapted in accordance with the device used for administration. For example, the composition can comprise a suitable buffer system to suitable pH, e.g., near neutral pH (e.g., phosphate or carbonate buffer system), and can comprise sufficient salt to ensure iso-osmotic conditions for the cells, i.e., preventing osmotic stress. For example, suitable solution for these purposes can be phosphate-buffered saline (PBS) as known in the art. Further, the composition can comprise a carrier protein, e.g., albumin, which can increase the viability of the cells. To ensure exclusion of non-human animal material, the albumin can be of human origin (e.g., isolated from human material or produced recombinantly). Suitable concentrations of albumin are generally known.

Hence, pharmaceutical compositions according to the present invention, and for use in accordance with the present invention, can comprise, in addition to the genetically-engineered cells, a pharmaceutically acceptable excipient, carrier, buffer, preservative, stabilizer, anti-oxidant or other material well known to those skilled in the art. Such materials should be non-toxic and should not interfere with the activity of the cells. The precise nature of the carrier or other material will depend on the route of administration. The composition can include one or more of cytoprotective molecules, such as the neuroprotective molecules as described herein, a neuro-regenerative molecule, a retinoid, growth factor, astrocyte/glial cells, anti-apoptotic factor, or factor that regulates gene expression in the cells of the invention. Such substances can render the cells independent of its environment.

A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. A pharmaceutically acceptable excipient, diluent, carrier and/or adjuvant as used in the specification and claims includes one and more such excipients, diluents, carriers, and adjuvants.

The invention also encompasses methods of producing said pharmaceutical compositions by mixing the cells of the invention with one or more additional components as above. Liquid pharmaceutical compositions generally include a liquid carrier such as water, petroleum, animal or vegetable oils, mineral oil or synthetic oil. Physiological saline solution, tissue or cell culture media, dextrose or other saccharide solution or glycols such as ethylene glycol, propylene glycol or polyethylene glycol may be included.

The composition can be in the form of a parenterally acceptable aqueous solution, which is pyrogen-free and has suitable pH, isotonicity and stability. Those of relevant skill in the art are well able to prepare suitable solutions using, for example, isotonic vehicles such as Sodium Chloride, Ringer's Injection, or Lactated Ringer's Injection. A composition can be prepared using biological fluids, such as artificial cerebrospinal fluid. In a further aspect, the invention relates to an arrangement comprising a surgical instrument for administration of a composition at a site of tissue dysfunction or lesion, such as the brain or tissue of the CNS, and further comprising the pharmaceutical composition as defined above, wherein the arrangement is adapted for administration of the pharmaceutical composition at the site of tissue dysfunction or lesion. For example, a suitable surgical instrument can be capable of injecting a liquid composition comprising the genetically engineered cells as described herein at the site of neural dysfunction or lesion. Cells can be implanted into a patient by any technique known in the art, including those described in Freed et al. 1997. Cell Transplant 6: 201-202; Kordower et al. 1995. N Engl J Med 332: 1 118-1 124; Freed et al. 1992. N Engl J Med 327: 1549-1555; Tateishi-Yuyama, Eriko, et al. The Lancet 360.9331 (2002): 427-435; THOMA, CHRISTINE, et al. Nature medicine 3.3 (1997); Kondziolka, D., et al. Neurology 55.4 (2000): 565-569, the entire disclosure of each which are incorporated herein by reference.

The pharmaceutical composition can further comprise at least one omega-3 fatty acid, such as those described herein. Non-limiting examples of omega-3 fatty acids include, 5,8,11,14,17-eicosapentaenoic acid (EPA), 4,7,10,13,16,19-docosahexanoic acid (DHA), 7,10,13,16,19-docosapentanoic acid (DPA), and α-linolenic acid (ALA).

Cytoprotective Conditioned Media

Embodiments of the invention comprise a conditioned media possessing the biological property of being neuroprotective. The conditioned media is characterized by being the product of culturing a plurality of genetically engineered cells as described herein in a media for a period of time so as to allow the cells secrete into the media neuroprotective factors.

The term “neuroprotective” can refer to the capability of a compound, such as a biological compound, or composition to prevent or reduce damage, including damage due to oxidative stress, of cells of the nervous system such as neurons and oligodendrocytes. In some embodiments the damage to the cells of the nervous system can be the result of a neurodegenerative disorder. In other embodiments the damage to the nervous system can be the result of an acute trauma. Non-limiting examples of neuroprotective protective factors comprise cytokines (such as IL-10), lipid mediators (such as NPD1, elavonoids and docosanoids), and exosomes (such as those derived from stem cells).

Media conditioned by genetically-engineered cells as described herein can comprise biological activities of the cells themselves, and are capable of substituting for the cells themselves. Therefore, the biological property or biological activity the genetically-engineered cells can correspond to a biological property or activity of the conditioned medium. Accordingly, the genetically-engineered cells can comprise one or more biological properties or activities the conditioned media.

The conditioned cell culture medium can be obtained by culturing genetically-engineered cells as described herein, a descendent thereof (for example, cells taken from a first, second, or third passage), or a cell line derived therefrom in a cell culture medium; and isolating the cell culture medium.

The conditioned medium can be used in therapy as is, or after one or more treatment steps. For example, the conditioned medium can be UV treated or filter sterilised. One or more purification steps can be employed. In particular, the conditioned media can be concentrated, for example by dialysis or ultrafiltration. For example, the medium can be concentrated using membrane ultrafiltration with a nominal molecular weight limit (NMWL) of for example 3K.

For the purposes described in this document, for example, treatment or prevention of disease, a dosage of conditioned medium comprising about 15 mg to about 750 mg protein/100 kg body weight may be administered to a patient in need of such treatment.

Methods of Treatment

The invention provides a method for treating a subject afflicted with a disease, such as an ophthalmological, neurological or neuropsychiatric disease. In embodiments, the subject afflicted with a disease can be referred to as a patient in need thereof.

Further, the invention provides for a method of reducing or ameliorating symptoms associated with a neurological disease, a method of reducing or ameliorating pathogenesis associated with a neurological disease, a method of delaying the onset and/or progression of a neurological disease, and a method of promoting neuronal recovery and restoration of neuronal function. For example, neuronal recovery and/or restoration of neuronal function can be measured by EEG or a neurological assessment.

Non-limiting examples of pathogenesis comprises neuroinflammation, neuronal death, neuronal injury, Aβ formation, infarct volume, and oxidative stress.

Compositions as described herein can promote neuronal survival, neuronal differentiation, neurogenesis, neurite growth, synaptogenesis, synaptic protein expression, synaptic function, which can be clinically measured by neurological functional tests, EEG, MRI, or a combination thereof.

DHA affects neurogenesis. For examples, DHA supplementation induces accelerated neurite outgrowth (L. Dagai et al., 2009), DHA enhances neuronal differentiation of neural stem cells (M. Katakura et al., 2009), DHA increases the number of neurons derived from neural stem/progenitor cells (Nobuyuki Sakayoti et al., 2011), and DHA promotes neurite growth, synaptogenesis, synaptic protein expression and synaptic functions in hippocamal neuron (Cao, D et al., 2009). Further, DHA increased neurogenesis by proliferation, differentiation and migration of neural stem cells in the DG, SVZ and peri-infarct area, and promoted neurobehavioral recovery and reduced infarct volume.

In some embodiments, neurogenesis is detected using 5-bromo-2′-deoxyuridine, Ki-67, DCX, NeuN, or a combination thereof 5-bromo-2′-deoxyuridine (BrdU) is an analog of thymine, and is incorporated into newly-synthesized DNA of replicating cells. Ki-667 is a market for mitotic cells. DCX is a marker for immature and early post-mitotic neurons. NeuN is a marker for mature neurons.

The term “therapeutically effective amount” can refer to that amount of an embodiment of the composition or pharmaceutical composition being administered that will relieve to some extent one or more of the symptoms of the disease or condition being treated, and/or that amount that will prevent, to some extent, one or more of the symptoms of the condition or disease that the subject being treated has or is at risk of developing.

Generally, the method comprises administering a therapeutically effective amount (i.e., an amount sufficient to elicit a local or systemic effect) of the genetically engineered cells or compositions comprising the same to a subject afflicted with the disease. For instance, a plurality of genetically engineered cells as described herein can be transplanted or injected into a subject, wherein the cells engraft within a tissue of the subject. Without wishing to be bound by theory, embodiments as described herein can restore homeostasis of the nervous system by reducing or ameliorating neuroinflammatory disturbances, which can contribute to the treatment, delay or prevention of the onset and/or progression of diseases as described herein.

For example, at least one of the plurality of genetically engineered cells administered to a subject can engraft within a tissue of the subject, such as within a tissue of the nervous system (i.e, the central nervous system, the peripheral nervous system, or a combination thereof). Non-limiting examples of tissues of the nervous system comprises the brain, spinal cord, optic nerve or combination thereof.

Neurological diseases that can be treated using the genetically engineered cells and compositions comprising the same include those diseases characterized by neuronal injury, neuroinflammation, neuronal dysfunction and/or neurodegeneration, neuronal damage or neuronal loss (i.e. cell death). In particular, such neuronal dysfunction, degeneration, damage or loss can be to cortical areas, and can affect one or more types of cortical pyramidal neurons or cortical inhibitory interneurons. Clinical assesments, such as neurological and/or ophthalmological assessments, for example, can provide information about functional recovery.

According to the invention, “symptoms” can refer to one or more biological and/or physiological sequelae, including but not limited to memory loss, personality changes, problems with movement, weakness, or poor balance or coordination. Some common symptoms of degenerative disorders of the brain are memory loss, personality changes, problems with movement, weakness, or poor balance or coordination.

“Neuronal injury” can refer to the damage to the function or structure (e.g., cytoskeletal damage) of neurons as a result of an insult (e.g., exposure to neurotoxins) or trauma (e.g., traumatic brain injury, concussive head trauma) to the nervous system. Neuronal injury is associated with, for instance: stroke, ischemic events (e.g., brain ischemia, ischemia of the eyes), seizures of diverse etiology (epileptic, associated with brain injury, of genetic origin), spinal cord injury or trauma, brain damage due to drugs of abuse, or excitotoxic insults of diverse nature.

In embodiments, the “neuronal injury” or “neuronal cell death” can be the result of a stroke. For example, “stroke” can refer to any acute, clinical event related to the impairment of cerebral circulation. The terms “acute cerebral ischemia” and “stroke” can be used interchangeably.

As used herein, “neurodegeneration” can refer to the progressive loss of individual or collective structure or function of neurons, up to and including the death of neurons that is associated with many neurodegenerative diseases.

For example, “neurodegenerative disease(s)” or “neurodegenerative disorder” can refer to medical conditions that are characterized clinically by their insidious onset and chronic progression. In many instances, particular parts of the brain, spinal cord, or peripheral nerves functionally fail and the neurons of the dysfunctional region die. Neuroanatomically localizable functional impairment and “neurodegeneration” associate with recognizable syndromes or conditions that are ideally distinct, although in clinical and even neuropathologic practice substantial overlap exists. Neurodegenerative diseases are often categorized by whether they initially affect cognition, movement, strength, coordination, sensation, or autonomic control.

Frequently, however, patients will present with symptoms and signs referable to more than one system. Either involvement of several systems can occur concomitantly, or else by the time the patient has functionally declined enough to seek medical attention multiple systems have become involved. In many cases, the diagnosis of a neurodegenerative disease cannot be critically ‘confirmed’ by a simple test.

The term “neurodegenerative” can refer to the loss of neurons that cause disease.

However, without being bound by theory, neuronal demise can be the final stage of a preceding period of neuron dysfunction. It is difficult to know whether clinical decline associates with actual neuron loss, or with a period of neuron dysfunction that precedes neuron loss. Also, particular neurodegenerative diseases are etiologically heterogeneous. In addition to syndromically defining neurodegenerative diseases by what neuro-anatomical system is involved, these disorders are broken down along other clinical lines. Early (childhood, young adulthood, or middle aged adulthood) versus late (old age) onset is an important distinction. Some clinically similar neurodegenerative diseases are sub-categorized by their age of onset, despite the fact that at the molecular level different forms of a particular disease may have very little in common. Sporadic onset versus Mendelian (genetic) inheritance constitutes another important distinction, and many named neurodegenerative diseases have both sporadic (wherein Mendelian inheritance is not recognizable) and Mendelian subtypes.

Non-limiting examples of neurodegenerative diseases comprise: dementia, for example Alzheimer's Disease, multi-infarct dementia, AIDS-related dementia, and Fronto temperal Dementia; neurodegeneration associated with cerebral trauma; Parkinson's Disease; Amyotropic Lateral Sclerosis (ALS); Multiple Sclerosis (MS); Huntington's disease; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; and multi-system atrophy.

Neurodegenerative diseases can present with memory loss or personality change, non-limiting examples of which comprise Alzheimer's disease, Frontotemporal Dementias, Dementia with Lewy Bodies, Prion diseases.

Neurodegenerative diseases can present as problems with movement, non-limiting examples of which comprise Parkinson's disease, Huntington's disease, Progressive Supranuclear Palsy, Corticobasal Degeneration, Multiple System Atrophy.

For example, neurodegenerative diseases can present as weakness, non-limiting examples of which comprise, amyotrophic lateral sclerosis, inclusion body myositis, degenerative myopathies.

Neurodegenerative diseases can also present as poor balance, non-limiting examples of which comprise the spinocerebellar atrophies.

Disorders of myelin include multiple sclerosis and Charcot-Marie-Tooth disease.

The term “motor neuron diseases” (MNDs) refers to a group of progressive neurological disorders that destroy motor neurons, the cells that control essential voluntary muscle activity such as speaking, walking, breathing, and swallowing. The best-known motor neuron disease is amyotrophic lateral sclerosis (ALS). Normally, messages from nerve cells in the brain (called upper motor neurons) are transmitted to nerve cells in the brain stem and spinal cord (called lower motor neurons) and from them to particular muscles. Upper motor neurons direct the lower motor neurons to produce movements such as walking or chewing. Lower motor neurons control movement in the arms, legs, chest, face, throat, and tongue. Spinal motor neurons are also called anterior horn cells. Upper motor neurons are also called corticospinal neurons.

When there are disruptions in the signals between the lowest motor neurons and the muscle, the muscles do not work properly; the muscles gradually weaken and may begin wasting away and develop uncontrollable twitching (called fasciculations). When there are disruptions in the signals between the upper motor neurons and the lower motor neurons, the limb muscles develop stiffness (called spasticity), movements become slow and effortful, and tendon reflexes such as knee and ankle jerks become overactive. Over time, the ability to control voluntary movement can be lost. The following is a list of the most common MNDs: Amyotrophic lateral sclerosis (ALS), also called Lou Gehrig's disease, progressive bulbar palsy, also called progressive bulbar atrophy, pseudobulbar palsy, Primary lateral sclerosis (PLS), progressive muscular atrophy, spinal muscular atrophy (SMA) and some of its variants (e.g., SMA type I, also called Werdnig-Hoffmann disease, SMA type II, SMA type III also called Kugelberg-Welander disease, congenital SMA with arthrogryposis, Kennedy's disease, also known as progressive spinobulbar muscular atrophy and post-polio syndrome (PPS)).

The cells of the present invention are administered and dosed in accordance with good medical practice, taking into account the clinical condition of the individual patient, such as the extent of the lesion or disease condition, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

In the method of the present invention, the cells of the present invention can be administered in various ways as would be appropriate to implant in the central nervous system, including but not limited to parenteral administration, intrathecal administration, intraventricular administration and intranigral administration.

In embodiments, the plurality of cells into which the transgene is introduced as described herein can be isolated from a subject to whom the genetically-engineered cells are ultimately to be reintroduced (i.e. personalized medicine approach). In an autologous transplantation of genetically engineered cells, for example, a subject's own cells, such as stem cells, are collected; a polynucleotide as described herein is then introduced into the subject's cells to make genetically engineered cells that express a receptor that transports an omega-3 fatty acid across the cell membrane; and the genetically engineered cells are then reintroduced back into the subject.

Where administration of the genetically engineered cells or compositions comprising the same to a patient is contemplated, it can be preferable that the cells are selected such as to maximize the tissue compatibility between the patient and the administered cells, thereby reducing the chance of rejection of the administered cells by patient's immune system (graft vs. host rejection). For example, advantageously the cell lines can be typically selected which have either identical HLA haplotypes (including one or preferably more HLA-A, HLA-B, HLA-C, HLA-D, HLA-DR, HLA-DP and HLA-DQ; preferably one or preferably all HLA-A, HLA-B and HLA-C) to the patient, or which have the most HLA antigen alleles common to the patient and none or the least of HLA antigens to which the patient contains pre-existing anti-HLA antibodies.

Embodiments as described herein can be used to treat, prevent, or delay the progression of neurological disorders, non-limiting examples of which comprise Alzheimer's disease, Huntington's chorea, Parkinson's disease, dementia, HIV dementia, stroke, epilepsy, ALS, multiple sclerosis, traumatic brain injury, cerebral ischemia, and cerebral hemorrhage.

Embodiments as described herein can treat, prevent, or delay the symptoms, pathogenesis, and/or progression of ophthalmolgocal disorders, non-limiting examples of which comprise retinal degenerative diseases (retinitis pigmentosa, age-related macular degeneration other retinal degenerative disease), glaucoma, and ischemic retinal disease.

Embodiments as described herein can be used to treat, prevent, or delay the symptoms, pathogenesis and/or progression of neuropsychiatric disorders, non-limiting examples of which comprise depression, mania (such as bipolar disorder), schizophrenia, visual hallucinations, obsessive-compulsive disorder, and eating disorders.

FIG. 5 describes one embodiment of the invention. The cell, which may be a stem cell or other cell, is stably transfected with a vector or expression cassette expressing the Adiponectin Receptor 1 gene, the membrane frizzled-related protein gene, or both. Expression of the transfected gene(s) is confirmed by Western blot analysis, enhanced ability of the cell to take up deuterium-labeled DHA, enhanced survival from oxidative stress. Once expression of the transfected gene(s) is confirmed, the genetically-engineered cell(s) can be administered to the patient. For example, the genetically-engineered stem cell can be injected into the brain of a patient diagnosed with disease (Lee I H, et al., Delayed epidural transplantation of human induced pluripotent stem cell-derived neural progenitors enhances functional recovery after stroke. Sci Rep. 2017 May 16; 7(1):1943; Li W. et al., Extensive graft-derived dopaminergic innervation is maintained 24 years after transplantation in the degenerating parkinsonian brain. Proc Natl Acad Sci USA. 2016 Jun. 7; 113(23):6544-9, both of which are incorporated by reference in their entireties). In one embodiment, the patient can be diagnosed or predisposed to Alzheimer's disease, or diagnosed or predisposed to Age-Related Macular degeneration. FIG. 7 describes the general biological pathway by which DHA delivery to the genetically engineered cells may sustain reversal of A-beta peptide formation in Alzeimer's disease and age-related macular degeneration.

### The Kit

The invention also provides for a kit for treating disease in a subject. Non-limiting examples of components of the kit comprise a therapeutically effective amount of genetically engineered cells as described herein, a composition comprising genetically engineered cells as described ere herein, instructions for use, or any combination thereof.

The kit can be used to carry out the methods as described herein, such as methods of treating a subject afflicted with a neurological disorder.

The genetically engineered cells can be packaged by any suitable means for transporting and storing cells. For example, the cells can be provided in frozen form, such as cryopreserved; dried form, such as lyophilized; or in liquid form, such as in a buffer. Cryopreserved cells, for example, can be viable after thawing.

The kit can include devices to administer the compositions, such as an injector to inject the composition into a subject.

The instructions generally include one or more of: a description of the genetically engineered cells; methods for thawing or preparing cells, dosage schedule and administration for treatment of a disease; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. Generally, a kit as described herein also includes packaging. In some embodiments, the kit includes a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding cells or medicaments.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Generation of Adipose Tissue-Derived Stem Cells (ASC) and ABCs Overexpressing MFRP and ADIPOR1

Adipose tissue-derived stem cells (ASC) were transduced with eGFP-MFRP lentivirus and as controls we used mCherry-ADIPOR1 lentivirus. On the other side ABC-mcherry-ADIPOR1 were transduced with eGFP-MFRP. Antibiotic selection was started 3 days after transduction only in the ASCs, and lasted at least three weeks.

Zeiss 710; same settings were used to capture ASCs-lentivirus (FIG. 1) with red and green separate channels. Here only MFRP-ASCs emitted green fluorescence and only ADIPOR1-ASCs emmitted red fluorescence, thus confirming the successful transduction. MFRP-ASCs may be expanded for ensuing western blot confirmation.

In the top FIG. 2, all cells emit strong red fluorescence indicating stable constitutive expression of ADIPOR1 under CMV promoter. Three weeks after secondary transduction with the MFRP-eGFP, the majority of the cells co-express red/green fluorescence with a Pearson's overlap coefficient equal to 74%. Therefore secondary transduction is feasible in the ABCs cells.

In order to determine the gain-of-function increased lipid uptake, ASCs-mCheryy and ASCs-mCherry ADIPOR1 (4 weeks post transduction/selection) were incubated in the presence of 0.1 μM DHA, during 3 hours. Liquid-liquid lipid extraction was performed and MS analysis performed. Results are shown on FIGS. 3-9.

In order to optimize the experimental conditions under which brain and stem cells uptake DHA to produce NPD1 exponentially growing rat glial cells (SVGs) were incubated in the presence of 1 μM during 1 or 6 hours before lipid extraction and MS analysis. Results are shown on FIGS. 10-13.

### Example 2

Generation of Cell Clones with Constitutive Expression of ADIPOR1 or MFRP and Co-Expressing ADIPOR1/MFRP in Human Primary Cells

Reagents and cells—For the generation of stable clones constitutively expressing ADIPOR1 (and/or MFRP) we utilize high titer lentiviral stocks acquired from Genecopeia (Rockville, Mass.). The plasmid constructs we selected to use contain a cassette for either mCherry (for ADIPOR1) or eGFP (for MFRP) and both contain a puromycin resistance cassette. As positive controls we utilize empty plasmids lentiviral stocks carrying the mCherry or the eGFP. Cells lines utilized are skin fibroblasts, adipose tissue stem cells, primary retinal pigment epithelial cells (ABC cell generated in our lab, paper to be published, other stem cells).

Rationale for the use of human ASCs—Human ASCs have been successfully xenotransplated into mice and rat models of neurodegenerative diseases (e.g. Human adipose-derived stem cells stimulate neuroregeneration. Masgutov R F, Masgutova G A, Zhuravleva M N, Salafutdinov I I, Mukhametshina R T, Mukhamedshina Y O, Lima L M, Reis H J, Kiyasov A P, Palotás A, Rizvanov A A. Clin Exp Med. 2015 Jun. 6) which demonstrates that human ASCs have low immunogenicity. As our studies are aimed into translational medicine, our preclinical studies in rat or mouse should be performed with the use of human ASCs rather than rodent isogenic murine or rat ASCs. Most surface cell markers for ASCs are species specific; genes for ADIPOR1 and MFRP are in part species specific, therefore we use human ASCs in vivo. We also use rat- or mouse-derived ASCs.

Overexpression of ADIPOR1 and MFRP in ASCs and RPEs—The generation of ASCs genetically engineered to possess increased ability to uptake DHA by incorporating in their genome ADIPOR1 or MFRP or both. Once injected in the injury site, ASC-DHA cells would present increased transdifferentiation potential into retinal pigment epithelial cells, photoreceptors, other retinal cells or brain cells accordingly.

Strategy—Obejective1: Generate stable transfectants expressing ADIPOR1-mCherry and clones expressing the control plasmids mCherry and eGFP.

To do that ˜2×104 (ASCs or ABCs) will be seeded in 6-well plates, let adhere overnight. The following day, media will be replaced with fresh 0.5 ml complete media containing serial dilutions of 10, 5, 2.5, 1.25 and 0.125 μls of lentiviral stock (one well with fresh media alone. One plate (3 in total) will be dedicated for each expression plasmid (ADIPOR1-mCherry, mCherry and eGFP). In parallel similar transduction will be done in optical-six well plates.

a. Endpoint Analysis:
i. Day 1 after transduction: Cell health/viability will be assessed by inspection. Transduction media will be discarded and replaced with fresh complete media containing 2 μg/ml puromycin. Media+puromycin will be changed every day until all cells are dead in the mock transduction well.
ii. Day 3 after transduction: Efficiency of transduction will be assessed by confocal microscopy and western blot analysis. We will utilize the optical-bottom plates for mCherry-red fluorescence measurements by confocal microscopy. Thereafter cells will be detached with trypsin-EDTA, processed for western blot analysis of ADIPOR1 expression using in house protocols and reagents.
iii. Day 10-20 after transduction: Surviving clones will be pooled together to present “clonal effect” in our studies. Specific transgene expression assessed by western blot, qRT-PCR and sequencing.
iv. Outcomes: Transduction efficiency here will be indicated as % cells mCherry positive, and by western blot densitometry analysis (% intensity transduction relative to control) should increase and be statistically significant in a dose-dependent manner.

Strategy—Objective 2: Generate stable clones co-expressing mCherry and eGFP following co-transduction of target cells with 1:1 amounts of mCherry:eGFP, final amounts 0.1, 1, 2, 5 and 10 μl/well, followed by antibiotic selection (puromycin) as above described. Likewise efficiency of transduction will be assessed with the confocal microscope and by bivariate analysis utilizing flow cytometry. Selected puromycin-resistant clones will be further selected by FACS, expanded and stocks generated. These clones should be available by mid-July.

Strategy—Objective 3: Generate stable clones expressing MFRP-eGFP-puromycin resistant, by using similar procedures as above described (Objective 1); generate target cells expressing MFRP and ADIPOR1 utilizing similar strategy as described (objective 2).

Upon receiving the reagents, generate within a short period (3 weeks) stable transfectants expressing MFRP-eGFP utilizing lenti:targets ratios based on the observations from objective1; generate clones of target cells co-expressing MFRP and ADIPOR1 by co-transducing target cells with equal amounts of MFRP-eGFP and ADIPOR1-mCherry viral stocks (objective 2). Following antibiotic selection, western blot analysis, chimeric clones MFRP-ADIPOR1 will be further selected by FACS, expanded and stocks generated.

Strategy—Objective 4: Assessment of DHA uptake by target cells constitutively expressing ADIPOR1 under CMV promoter.

These experiments will be carried out utilizing 6-well plates. For the first series of experiments incorporation of DHA-d5 followed by mass spectrometry lipidomic analysis (Adiponectin receptor 1 conserves docosahexaenoic acid and promotes photoreceptor cell survival. Rice D S, Calandria J M, Gordon W C, Jun B, Zhou Y, Gelfman C M, Li S, Jin M, Knott E J, Chang B, Abuin A, Issa T, Potter D, Platt K A, Bazan N G. Nat Commun. 2015 Mar. 4; 6:6228. doi: 10.1038/ncomms7228. PMID:25736573), transfectants utilized will be mCherry-vector, ADIPOR1-mCherry; scramble and ADIPOR1 siRNA in addition to mock transfection cells. In the second phase these studies will include the MFRP transfectants and MFRP_AIPOR1 transfectants.

Test the Use of Transfectants
Transdifferentiation of human ADIPOR1-, MFRP- and ADIPOR1-MFRP-ASC in vitro and in vivo
Survival and lipidomic response to oxidative stress
TBD (e.g. NPD1 and DHA uptake by ADIPOR1)
Generate lentivirus plasmid ADIPOR1-MFRP chimeras

### Example 3

DHA-d5 and AA-d8 Uptake by Human Adipose Tissue-Derived Stem Cells (hASC) and by Human Primary Retinal Pigmented (ABC) Cells Overexpressing ADIPOR1 Transgene

Background and significance—Although it is known that ADIPOR1 gene function play important role in the specific cellular uptake and retention of DHA, the intracellular mechanisms regulating this process are unknown. DHA, which is indispensable for the production of stress-induced cytoprotective molecule NPD1, is deficient in the brain during neurodegenerative diseases. Injected stem cells (SCs) have tropism to injured areas such as the brain after stroke. In these locations SC replace damaged and dead cells following transdifferentiation into neural cells. Therefore, SCs overexpressing ADIPOR1 transgene could accomplish simultaneously two functions:

1—Increase NPD1 availability in damaged areas following DHA-target delivery

2—Accelerate tissue healing.

Strategy 1—ADIPOR1 in concert with MFRP increases specific DHA cellular uptake.

Aim 1: Determine increased ability of ADIPOR1-ASCs to uptake and retain DHA.

### Experiment 1: Compare DHA-d5 vs AA-d8 Uptake

Two separate cultures of exponentially growing mCherry- or ADIPOR1-hASC were used seeded into two separate 6-well plates each at the density of 1×10⁶ cells per well, and let adhere during 48 hours (5% CO₂ and 95% air humidified atmosphere). Similar setup was made with the ABCs. Next regular media was replaced with complete media (without antibiotics) and 3% w/v fatty acid free BSA and cells incubated for another 24 hours. Thereafter media was replaced with complete BSA media plus vehicle (ethanol 0.01% v/v) or complete BSA media plus 1 μM each DHA-d5 and AA-d8 and plates incubated for an additional 24 hours. Next plates were placed in ice, media aspirated, wells rinsed three times

with ice-cold PBS and cells scrapped in 1 ml MeOH-BHT, procedure was repeated 2 more times with ice-cold MeOH before processing for lipid extraction followed by hydrolysis and mass spec analysis.

Results—Human ASC overexpressing Adipor1 had significantly higher levels of DHA-d5 vs control (p=0.0035 and 0.0012, with t-test). AA-d8 uptake was not significant experiment 1 (p=0.0619) and significant experiment 2 (p=0.0114).

ABC cells overexpressing Adipor1 had significantly higher levels of DHA-d5 vs control (p=0.0067 and 0.0428, with t-test). AA-d8 uptake was statistically significant (p=0.0260) experiment 1 and not significant experiment 2 (p=0.0915).

Although we used 1 μM each AA-d8 and DHA-d5, uptake of the later was ˜10 and ˜20 times higher than that of AA-d8 respectively in the ADIPOR1-ABC and ADIPOR1-ASCs.

In summary these results indicate that there might be differences of uptake between cell ASC and ABC types and that DHA-d5 is uptake preferentially over AA-d8 by these cells.

### Experiment 2—Determine ADIPOR1-ASCs In Vitro Ability to Delivery DHA-d5 to Stroke-Like Injured Tissue

ABCs were seeded in 6-well plates 0.5×10⁶ cells/well, 48 hours later cells regular media was replaced with 1 ml of glucose free DMEM containing 1% FBS and incubated in normoxic environment during 2 hours (@37° C., 95% air, 5% CO₂ humidified atmosphere). than during 1 hour in hypoxic humidified atmosphere (@37° C., 95% N₂, 5% CO₂). Next mCherry- or ADIPOR1-ASCs previously incubated during 24 hours with 1 μM DHA-d5 or 0.01% v/v ethanol, were washed three times with sterile PBS trypsinized, resuspended at the density of 0.1×10⁶ cells/ml of complete medium with 10% FBS, and 2 ml added to each well of injured cells as duplicates. Lipid extraction and MS measurement of DHA-d5 content will be performed in the media collected and adherent cells. Media will be collected every 2 days during one week culture, centrifuged 42000 rpm, 30 minutes and clear media stored at −80 C until analysis.

### Experiment 3—Determine the Influence of DHA in the Transdifferentiation of ASCs into ABCs

ABCs were seeded in cover slips in 6-well plates let adhere during 48 hours before being processed as in experiment 2.

Results—Both MIFT and RPE65 immnunostainings were done directly on 6-well plates. Poor quality IF detection could be do thickness and lower magnification, therefore we are now growing cells in coverslips. Another alternative to improve signal intensity and reduce high noise would be using amplification with tyramide conjugates with gains of signal >200 folds.

Alternative Approach—

1. Following binding to its cognate ligand, ADIPOR1 activates AMPK pathway which subsequently leads to increased specific DHA uptake.

2. Other promoters such as E2F1 could be more efficient in expressing ADIPOR1 and MFRP transgenes in stem cells

### Experiment 4—Determine Adiporon Dose-Dependent DHA-d5 Uptake by ADIPOR1-hASC

Pre-incubation of ADIPOR1-ASCs with ADIPOR1 agonists will enhance their ability to uptake and retain DHA mCherry and ADIPOR1-ASC will be co-incubated 1 μM DHA-d5 plus various concentrations of adiporon 0.5-50 μM during 24 hours as triplicates at each concentration. Thereafter adherent cells will be used for lipid extraction and MS analysis of DHA-d5 uptake.

Figures

1. Exponentially growing hASC (bright field) and confocal microscopy

2. WB and densitometry analysis of ADIPOR1 expression in ABCs and ASCs

3. DHA-d5 and AA-d8 uptake by ABCs and ASCs

4. Immunofluorescence staining of MITF and RPE 65 in ABC/ASCs cocultures

### Example 4

Genetically-Engineered Stem Cell or Other Cells to Retain Omega-3 Fatty Acids

This invention centers around using genetically-engineered stem cells or other cells to express the integral membrane protein adiponectin receptor 1 (AdipoR1} to mediate docosahexaenoic acid (DHA) cellular uptake and retention.

AdipoR1 is the receptor for the hormone adiponectin, which regulates insulin sensitivity, has anti-inflammatory actions, and is a pro-cell survival effector. We discovered that this receptor specifically captures and retains DHA and that it is necessary for cell survival (Rice D S, Calandria J M, Gordon W C, Jun B, Zhou Y, Gelfman C M, Li S, Jin M, Knott E J, Chang B, Abiun A, Issa T, Potter D, Platt K A, Bazan N G, Adiponectin receptor 1 conserves docosahexaenoic and promotes photoreceptor cell survival, Nature Communications, 2015).

The cognate ligand, adiponectin, is not involved in the ability of the AdipoR1 to facilitate DHA cellular uptake. The cells comprise at least one gene that expresses the AdipoR1 that, in turn, has the selective ability to take up and retain DHA. The following cells can be genetical engineered: stem cells derived from adipose tissue, bone marrow, blood, human brain cells, pericytes, macrophages, retinal pigment epithelial cells, and others.

This invention relates to therapeutic combinations, genetically-engineered cells; compositions, and their methods of use for the treatment of neuroinflammatory/immune conditions, including Alzheimer's disease; ischemic stroke, epileptogenesis, traumatic brain injury, spinal cord injury, age-related macular degneration,! Parkinson's disease, multiple sclerosis (MS), amyotrophic lateral sclerosis (ALS}, and other neurological and ophthalmological diseases as well as other conditions such as osteoarthritis, metabolic syndrome, other chronic diseases and tumors.

DHA supplementation induces the accelerated neurite outgrowth (L. Dagai et al., 2009)

DHA enhances neuronal differentiation of neural stem cells (M. Katakura et al., 2009)

DHA increases the number of neurons derived from neural stem/progenitor cells (Nobuyuki Sakayoti et al., 2011)

DHA promotes neurite growth, synaptogenesis, synaptic protein expression and synaptic functions in hippocamal neuron (Cao, D et al., 2009)

### Example 5

Genetically-Engineered Stem and Transdifferentiated Cells for Neurorestration in Stroke

There are no effective therapies (including stem cell therapies) for neurological diseases resulting in damage and neuronal/brain cell loss. This project is based upon the notion ischemic-stroke triggers damaging neuroinflammation and oxidative stress that enhances damage and prevents stem cell survival and engraftment.

We discovered proteins that capture precursors of bioactive mediators that counteract these actions, two of which determine DHA uptake and cell survival: AdipoR1 and MFRP. We will conduct studies assessing genetically-engineered stem cells expressing proteins that facilitate repair and neurorestoration after experimental ischemic-stroke, characterizing transdifferentiated skin fibroblasts with lentivirus-expressing mCherry, eGFP protein controls, or other genes.

Our genetically-engineered cells that express proteins for selective

DHA uptake offer the opportunity to replace lost/damaged neurons as well as to counteract homeostatic disruptions and neuroinflammation. The proteins enhance DHA delivery to cells and, in turn, activate the conversion of the fatty acid into bioactive docosanoids. Overall, the sustained uptake, retention and DHA availability lead to docosanoid synthesis made on-demand, in turn, resulting in cell protection and disease reversal by halting and/or reversing damage and eliciting function restoration. Our recent paper demonstrates that one of the proteins that we now succeeded in expressing in our GESC, the Adiponectin receptor 1 (AdipoR1) protein, is necessary for cell function by capturing DHA (6). We recently discovered that the other protein that we are expressing in our GESC, membrane-type frizzled-related protein (MFRP), is also involved in DHA uptake.

The validation studies will test two new lines of genetically-engineered stem cells (GESC) as potential lead therapeutic agents. The two lines are (i) skin fibroblast trans-differentiated into neurons or (ii) adipose tissue derived stem cells. Both lines of GESCs will be genetically engineered to express two proteins that selectively capture DHA into cells, the AdipoR1 protein and MFRP. These proteins will allow the GESCs to capture more DHA, which will counteract neuroinflammation, promote circuit integrity, and replace lost neurons (5). We will explore if one or both GESC lines are effective in neuroprotection and neurorestoration in the ischemic stroke model.

Successful completion of these studies will demonstrate neurological recovery, validated by key cellular and molecular markers of neuroprotection. Without wishing to be bound by theory, these studies will demonstrate the improved protection and survival of neurons in the penumbra, as well as restoring of neurons into neurocircuitry. For example, restoration of neuronal function can be measured by EEG or a neurological assessment.

Taken together, the successful completion of these studies will not only validate this unique concept as a potential therapeutic agent to treat neurodegenerative diseases, but will also serve as the foundation of future marketing efforts for this technology.

Explanation of Specific Experiments and Concepts to be Validated

Our validation studies will test a novel therapeutic concept for neurodegenerative diseases using a physiologically relevant ischemic stroke animal model. The therapeutic products under development are genetically engineered stem cells (GESCs). The GESCs described in the validation studies are of two kinds: a) skin fibroblasts transdifferentiated into neurons; and b) adipose tissue-derived stem cells.

Several lines of research form the foundation of these studies. First, we have successfully developed these cells and have found culture media conditions for neural differentiation (FIG. 3). Secondly, we have discovered that the adiponectin receptor 1 protein (AdipoR1) is necessary

for DHA uptake and cell function (1). Third, we have also identified the protein, MFRP, as actively engaged in DHA uptake. Finally, we have succeeded in stably expressing both of these proteins in fibroblasts using lentivirus constructs (FIG. 4). Taken together, the concept is that the neurons that evolve from GESC treatment will display an enhanced ability to capture DHA due to expression of AdipoR1 and MFRP, which in turn will set in motion pro-homeostatic signaling (FIG. 4).

For the testing of this, we chose the physiologically relevant ischemic stroke animal model because it triggers neuroinflammatory signaling (2) that thwarts neurorepair, such as uncompensated oxidative stress. Repair includes neuroprotection as well as the triggering of adult neurogenesis. The factors that induce, guide and sustain adult neurogenesis after injury are not understood. However, the substantial spontaneous recovery (weeks/months following injury) may involve at least, in part, some form of neurogenesis (3). Therefore, without wishing to be bound by theory, the GESCs will facilitate repair, replace irreversible damaged cells and, along with angiogenesis, lead to reparative responses after stroke.

Many types of stem/progenitor cells from different sources have been explored for their feasibility and efficacy for the treatment of stroke (4). Transplanted cells not only have the potential to replace lost circuitry, but also produce growth and trophic factors or stimulate the release of such factors from host brain cells, thereby enhancing endogenous brain repair processes. Although stem/progenitor cells have been shown to have a promising role in ischemic stroke in experimental studies as well as initial clinical pilot studies, cellular therapy is still at an early stage in humans (5). Many critical issues need to be addressed, including dose-response, therapeutic time window, cell type selection, and in vivo monitoring of their migration pattern. Our approach is different than those used up to now because GESC are endowed with key proteins.

Without wishing to be bound by theory, injection of the GESC in combination with DHA will lead to brain repair and neurorestoration after experimental ischemic stroke.

To explore this, we will complete the following experiments:

### Experiment 1: Study to Evaluate the Therapeutic Efficacy of GESC on Behavior and Infarct Size after Ischemic Stroke

Study Design: Male Sprague Dawley (SD) rats (280-300 g) will be anesthetized with isoflurane and subjected to 2 hours of middle cerebral artery occlusion (MCAo) (6). Physiological monitoring (cranial and body temperatures, blood pressure, blood gases, plasma glucose and hematocrit) will be measured before, during and after surgery. On day 1 after stroke, skin fibroblasts (SF) or adipose tissue (AT) (500,000 cells in 100 μl), or control (cerebral spinal fluid; CSF, 100 μl) will be administered into the lateral cerebral ventricle (icy) and DHA (5 mg/kg) or saline will be given intravenously (iv) (FIG. 5). I want to clarify that DHA will be injected systemically. We found that DHA systemically injected is neuroprotective against ischemic stroke in rats (8). In FIG. 1, we show the DHA administered icy is protective, which indicates that the enhanced availability in the parenchyma also elicits protection. Without wishing to be bound by theory, the combination of GESC administered icy with systemically injected DHA will be a future therapeutic approach.

Method for injection of GESC: Thirty minutes after removal of the suture, a brain infusion cannula will be implanted into the right lateral ventricle for treatment administration. Briefly, rats will be anesthetized with 3% isoflurane and will be secured to a stereotaxic apparatus with the skull leveled between the bregma and lambda. A sterile stainless steel cannula (5-mm long) will be implanted into the lateral ventricle using the stereotaxic coordinates (0.2 mm caudal to bregma, 2 mm lateral to midline, and 5 mm below the dura). Cannula will be removed after treatment is completed.

Animals will be allowed to survive for 8 weeks. In behavioral studies, the composite 12-point neuroscore will be performed weekly and rota-rod, Y-maze and beam walking test will be conducted on weeks 3, 4, 5, 6, 7 and 8 after MCAo. Magnetic resonance imaging (MRI) of the brains (7) and immunostaining will be used to analyze effect of GESC/DHA in the ischemic penumbra on weeks 3, 5 and 8 after MCAo. Different markers will be used to track GESC in the brain: neurons (NeuN and doublecortin), astrocytes (GFAP), microglia (ED-1) and SMI-71 immunopositive vessels after stroke (8).

Outcome: Without wishing to be bound by theory, combined SF/DHA or AT/DHA treatments will accelerate long-lasting behavioral recovery and protect the brain from severe damage caused by MCAo by reducing infarct size.

### Experiment 2: We Will Determine the Neuroprotective Dose-Response for the Treatment of Experimental Stroke with GESC (SF or AT) and DHA

Study Design: Rats will be prepared and subjected to 2 hours of MCAo (see Exp. 1). The following groups with different quantities of cells injected will be studied: 1-2) SF or AT, 300,000; 3-4) SF or AT, 400,000; 5-6) SF or AT, 500,000; 7-8) SF or AT, 600,000; 9-10) SF or AT, 700,000; and 11) control rats will be administered CSF (icy) day 1 after stroke. DHA will be administered (iv) in each group in addition to SF or AT on the same day.

### Experiment 3: We Will Determine the Therapeutic Window for the Treatment of Experimental Stroke with GESC (SF or AT) and DHA

Study Design: Rats will be prepared and subjected to 2 hours of MCAo (see Exp. 1). The therapeutic window will be defined using the best dose of SF/DHA or AT/DHA found in Experiment 2 to confer maximum neuroprotection. All treatments will be administered on days 1, 2, 3, 4 or 5 after stroke. Control rats will be injected CSF (100 μl) and saline on day 3 after stroke.

Analytical Methods and Data Analysis (Experiments 2 and 3): In each of the above protocols, neurological function will be assessed on the 12-point neurobehavioral battery (6) by wakening animals during MCAo (to verify the presence of a high-grade initial deficit) and on days 1, 2, 3 and 7 after reperfusion. Animals will be sacrificed 4 weeks after stroke and infarct volume will be calculated as we previously described (6). Analysis will be done blindly with respect to the experimental condition.

Outcome (Experiments 2 and 3): Without wishing to be bound by theory, SF/DHA and/or AT/DHA treatments will accelerate long-lasting behavioral recovery with an extended therapeutic window of up to 4 days after stroke and to reduce brain lesion from severe damage caused by MCAo.

Long-term potential. Our long-term goal is to establish a novel treatment for ischemic stroke and other neurodegenerative conditions by administration of GESC. Without wishing to be bound by theory, this GESC treatment will accelerate long-lasting behavioral recovery and protect the brain from severe damage caused by MCAo. Thus, without wishing to be bound by theory, the GESC treatment will have potential for the effective treatment of ischemic stroke and other diseases in patients.

Measurable milestones. The measureable milestones are:

Phase 1: To compare the effectiveness to protect/restore neurological function of two GESC lines in an experimental model of ischemic stroke. Specifically, neurological recovery will be assessed in phase 1.

Phase 2: To investigate the time-window for the administration of the most effective GESC.

Phase 3: To define cellular/neural circuitry restoration of the most effective GESC.

Metrics of success or failure. We will define the effectiveness of administration of two types of GESC in protecting and restoring ischemic-stroke damaged brain cells, a model of neurodegeneration. Achieving the measurable milestones herein will provide validation of the embodiments presented herein.

### References Cited in this Example

### References (for Description of Planned Work Section)

1. Rice D S, Calandria J M, Gordon W C, Jun B, Zhou Y, Gelfman C M, Li S, Jin M, Knott E J, Chang B, Abuin A, Issa T, Potter D, Platt K A, Bazan N G. Adiponectin receptor 1 conserves docosahexaenoic acid and promotes photoreceptor cell survival. Nat Commun. 2015; 6:6228. PMID: 25736573
2. Bazan NG1, Molina M F, Gordon W C. Docosahexaenoic acid signalolipidomics in nutrition: significance in aging, neuroinflammation, macular degeneration, Alzheimer's, and other neurodegenerative diseases. Annu Rev Nutr. 2011; 31:321-51. PMID: 21756134
3. Koh S H, Park H H. Neurogenesis in Stroke Recovery. Transl Stroke Res. 2016 (in press). PMID: 26987852
4. Ryu S, Lee S H, Kim S U, Yoon B W. Human neural stem cells promote proliferation of endogenous neural stem cells and enhance angiogenesis in ischemic rat brain. Neural Regen Res. 2016; 11:298-304. PMID: 27073384
5. Nagpal A, et al. TOOTH (The Open study Of dental pulp stem cell Therapy in Humans): Study protocol for evaluating safety and feasibility of autologous human adult dental pulp stem cell therapy in patients with chronic disability after stroke. Int J Stroke. 2016. pii: 1747493016641111. PMID: 27030504
6. Belayev L, et al. Middle cerebral artery occlusion in the rat by intraluminal suture. Neurological and pathological evaluation of an improved model. Stroke. 1996; 27:1616-22.
7. Obenaus A, Dilmac N, Tone B, Tian H R, Hartman R, Digicaylioglu M, Snyder E Y, Ashwal S. Long-term magnetic resonance imaging of stem cells in neonatal ischemic injury. Ann Neurol. 2011; 69:282-91. PMID: 21387373
8. Eady T N, Belayev L, Khoutorova L, Atkins K D, Zhang C, Bazan N G. Docosahexaenoic acid signaling modulates cell survival in experimental ischemic stroke penumbra and initiates long-term repair in young and aged rats. PLoS One. 2012; 7:e46151. PMID: 23118851

### Example 6

### Human Neurons Trans-Differentiated from Fibroblasts Overexpressing Adipor1 and MFRP Rescue Cortical or Hippocampal Neurons after Oxygen/Glucose Deprivation

Generation of Rat Primary Neurons.

All surgical procedures to isolated cortical and hippocampal primary neurons were performed under sterile conditions in a Nuaire Laminar Flow hood (Nuaire, Plymouth, Minn.). Timed pregnant embryonic day 18 (E18) Sprague Dawley rats (Charles River Laboratories) were euthanized and embryos harvested, sacrificed and brains aseptically collected and placed in petri dish containing ice-cold Hanks' Balanced Salt Solution, 1× (HBSS, #21-020-CV, Mediatech). Cortices and hippocampus were surgically isolated under a Nikon SMZ 645 stereomicroscope (Nikon, Melville, N.Y.). Tissues were chopped into small pieces with the help of micro-spring scissors, transferred to 15 ml Corning CentriStar centrifuge tubes (#430791, Corning, Corning, N.Y.). Next, HBSS containing 0.25% v/v trypsin (#15090-046, Gibco) and 0.25% w/v deoxyribonuclease I from bovine pancreas (DNase I, #DN25, SIGMA, St. Louis, Mo.) was added, then tubes placed in a water bath with agitation. After 15 minutes, FBS was added to 10% v/v (final concentration) to stop digestion. These tissues were subsequently triturated 15 times with a fire polished Pasteur pipet and left 2 minutes to allow debris to settle down. Supernatant was transferred to a new 15 ml centrifuge tube (Corning), then filtered through a 70 μm pore-sized Corning cell Strainer (#431751, Corning) and cells pelleted in a refrigerated Eppendorff 5840 R centrifuge (Eppendorff, Hauppauge, N.Y., 5 minutes, 1000 rpm at 4° C.). Cell′ pellets were resuspended in Neurobasal medium (GIBCO) supplemented with 2% v/v each B-27 and N2, along with 1× each Glutamax (#35050-061, Gibco) and penicillin/streptomycin (#15070063, Gibco). Cells were counted using a Neubauer hemocytometer under 10× objective utilizing a Nikon Eclipse TS100 microscope (Nikon). Cortical and hippocampal neurons were seeded at the densities of ˜2.5×10⁵ and ˜1.5×10⁵ cells/well respectively on TPP® tissue culture 12-well plates pre-coated with 25 μg/ml poly-L-lysine hydrobromide as described above and incubated at 37° C., 5% CO₂ humid atmosphere. The following media was aspirate and replaced with fresh pre-warmed complete neurobasal media. Subsequently, every three days half of the media was replaced with fresh medium.

Neuronal Immunophenotyping.

Neuronal and stem cells cultures were characterized by immunofluorescence staining with the class-III-β tubulin monoclonal antibody, the rabbit anti-glial fibrillary acidic protein, the doublecortin (#T8660 and #G9269, # D9818 all from SIGMA). In addition we used the Millipore monoclonal antibodies anti-NeuN (#MAN377) and anti-Map2 (#MAB3418) and the the rabbit monoclonal antibodies anti-synapsin (#5297) and synaptophysin (#5461) all purchased from CellSignal. Bound primary antibodies were detected following incubation with AF488 conjugated donkey anti-mouse IgG (#A-21202, Thermofisher) and AF647 conjugated donkey anti-rabbit (#A-31573) and nuclei counterstained with Hoechst 33258 (#94403, SIGMA).

Oxygen Glucose Deprivation (OGD).

In order to mimic stroke in vitro, two weeks-old rat neuronal primary cultures monolayers were washed with pre-warmed phosphate-buffered saline and pre-incubated in the presence of glucose-, sodium pyruvate- and glutamine free Neurobasal medium (#A24775-01, Gibco) during 30 minutes. Thereafter, plates were placed in a modular incubator chamber (#MIC-101, Billups-Rothenberg, Del Mar, Calif.) and air was replaced with an anaerobic mixture of gases (95% N₂, 5% CO₂, #1956, Air Gas, Radnor, Pa.) before placing in the incubator at 37° C. and anaerobic humid atmosphere.

In Vitro Cell-Based Rescue of Rat Primary Neurons from Programmed Cell Death after Insult by Nutrient and Oxygen Deprivation.

One hour after OGD, cells were returned to complete neurobasal medium alone, or with inserts with trans-differentiated neurons co-transduced with lentivirus control expressing mCherry and GFP or Adipor1 and MFRP proteins as above described or incubated in the presence or 1:1 NB complete:conditioned media before being placed in normoxic humid atmosphere with 5% CO₂.

Assessment of Cell Viability.

Three days after OGD, were applicable inserts were removed to new empty 12-well plates and fixed with 3.7% w/v formalin (#F79P-4, Fisher Scientific, Fair Lawn, N.J.) sealed and placed at 4° C. until further analysis. Next AlamarBlue® Cell Viability Reagent (#DAL1100, Life Technologies) was added (10% v/v). Rates of reduction of non-fluorescent blue-colored resazurin into highly fluorescent red-colored resorfurin were measured in a Spectramax M5 plate reader (excitation 530 nm/emission 590 nm, Molecular Devices, Sunnyvale, Calif.). For fluorescence readings of, two hundred microliters of media samples were collected and transferred from the experimental wells into Corning™ 96-Well Clear Bottom Black Polystyrene Microplate (#3603, Corning).

Statistical Analysis.

Experimental values are expressed as mean±SD from triplicates and significance was set at p value ≤0.05 with the one sided Student's t-test.

Results

FIG. 1 (A and B) shows stem cells issued from fibroblasts overexpressing Adipor-1 and MFRP display more far more mature morphology with conspicuous network of dendrites compared to their mCherry-GFP controls. These findings indicate that constitutive overexpression of Adipor1 and MFRP could induce greater DHA uptake and retention and subsequent production of DHA/EPA derivatives and/or the production of exosomes that are trophic and accelerators of stem cell's differentiation and maturation.

The rat primary neuronal cultures we used expressed the typical neuron-type biomarkers as shown in FIG. 1C. The Z′ factor for the alamarBlue assay was ≥0.9 which demonstrates that this assay is highly adequate for our experiments. As shown in FIG. 2, we observed that in the absence of insult, in the cortical neuron cultures, when compared to cells grown in regular complete neurobasal media, the cell numbers in the wells were not modified significantly, except by in the combination conditioned media plus cells in the BM-Adipor1-MFRP: here there was a slight, but statistical significant augmentation of cell metabolism. In the hippocampal neuronal cultures, condition media issued from cells transduced with the control lentivirus (mCherry-GFP) there was a minor decrease in the cell numbers of un-injured cells that is statistically significant. Similar to cortical neurons, BM media from Adipor1-MFRP transduced cells also induced great and statistically significant increase in relative cell numbers. It is possible that BM conditioned media from Adipor1-MFRP stem cells generates DHA/EPA derived factors and/or exosomes. These lipid derived factors might either (i) provide a relative growth advantage for the non-neuronal supporting cells in the culture or (ii) support mitochondrial biogenesis and/or (iii) enhances mitochondrial respiration in those trans-differentiated stem cells. Overall the biological significance of these differences is statistically significant. In summary, baseline viability is not unaffected by lentiviral transduction per se.

Three days following insult of neuronal cultures by uncompensated oxidative stress induced by nutrient and deprivation (OGD, FIG. 3), cell populations in the untreated control presented a robust and statistically significant near 50% reduction of viability relative to non-injured untreated controls in neurons issued both from cortex or hippocampus. Control neurons treated with mCherry/GFP conditioned media also showed similar levels of cell death as their non-transduced counterpart (differences not statistically significant between the injured cells). However in all conditions where cells were overexpress constitutively Adipor1 and MFRP we observed a sustained and statistically significant increase in neuronal cells resistance to OGD-induced cell death (FIG. 3). We found in both cortical and hippocampal neurons that after OGD, in the group of cells treated with conditioned media issued from neurons trans-differentiated from human fibroblasts overexpressing constitutively Adipor1/MFRP cell viability was increased relative to their mCherry/GFP counterparts in a statistically significant manner irrespective of either using IM or BM conditioned media. In the cortical neurons, the treatment of rat neurons after OGD with BM-conditioned media issued from Adipor1/MFRP overexpressing neurons transdifferentiated from human fibroblasts produced much greater survival than all other conditioned-media treated neurons which was statistically significant. In other words, BM conditioned media seems to be more cytoprotective than IM media and conditioned media from BM-Adipor1/MFRP is the most protective of all media. This observation per se may indicate that the sole presence of the activities of Adipor1/MFRP enabled transdifferentiated cells to generate additional neuroprotective factors to the ones provided by BM alone as previously mentioned. These observations warrant the need to increase statistical power of these experiments by repeating them in conjunction with lipidomic analysis of the media and cells. The data from the hippocampal neurons although indicating similar trend there are strong dissimilarities: IM-conditioned media from Adipor1/MFRP transduced neurons possessed the highest neuroprotection similar to that from the co-cultures with Adipor1//MFRP neurons. Furthermore there was a lost of neuroprotection by BM in the co-cultures with mCherry/GFP. It is too early to speculate whether or not these differences translate differences in the types of neurons issued from the cortex and hippocampus, but we may have to keep that into consideration.

In summary the system we used is adapted for the investigation of factors capable of providing neuroprotection after we mimic stroke in vitro. More importantly, these results demonstrate the potential in cell therapy of Adipor1/MFRP stem cells.

### Example 7

SEQ ID 1—Homo sapiens membrane frizzled-related protein (MFRP)

[symbol]

AAGGACTTCTCAGATGTCATCCTCTGCAT
GGAGGCAACAGAATCGAGCAAGACCGAGTTCTGCAATCCTGCCTTCGA
GCCTGAGTCTGGGCCACCCTGCCCTCCCCCAGTTTTCCCAGAGGATGC
CAGCTACAGCGTCCCAGCTCCCTGGCATGGTCGGCGTCCTCGAGGGCT
ACGGCCAGACTGCCGCTTCTCCTGGCTCTGTGTCCTCCTGCTCTCCAG
CCTGCTCCTCCTGCTGCTTGGGCTGCTGGTGGCCATCATCCTGGCCCA
GCTGCAGGCTGCACCCCCATCTGGGGCGTCCCATAGCCCACTGCCTGC
CGGAGGCCTTACCACGACCACCACCACCCCCACCATCACCACCTCTCA
GGCAGCTGGGACCCCTAAAGGGCAGCAGGAGTCAGGCGTGAGCCCCTC
CCCACAGTCCACCTGTGGAGGCCTCCTCTCTGGCCCAAGGGGCTTCTT
CAGCAGCCCTAACTACCCAGACCCTTACCCCCCCAACACCCACTGCGT
GTGGCATATCCAGGTGGCCACAGACCATGCAATACAGCTCAAGATCGA
AGCCCTCAGCATAGAGAGTGTGGCCTCTTGCCTTTTTGATCGCTTGGA
ACTCTCCCCTGAGCCTGAAGGCCCCCTCCTCAGGGTTTGTGGAAGGGT
GCCTCCCCCCACGCTCAACACCAATGCCAGCCACCTCCTGGTGGTCTT
CGTCTCTGACAGCAGTGTGGAAGGATTTGGTTTCCATGCCTGGTACCA
GGCTATGGCCCCTGGGCGCGGGAGCTGTGCCCATGATGAGTTCCGCTG
TGACCAGCTCATCTGCCTGCTACCTGACTCAGTGTGTGATGGTTTTGC
CAACTGTGCTGACGGCAGTGATGAGACCAATTGCAGTGCCAAGTTCTC
GGGGTGTGGGGGGAATCTGACTGGCCTCCAGGGCACTTTCTCTACTCC
CAGCTACCTGCAGCAGTACCCTCACCAACTGCTCTGCACCTGGCATAT
CTCGGTGCCTGCCGGACACAGCATAGAACTACAGTTCCACAACTTCAG
CCTGGAGGCTCAGGACGAGTGCAAGTTTGACTACGTGGAGGTGTATGA
GACCAGCAGCTCAGGGGCCTTCAGCCTCCTGGGCAGGTTCTGTGGAGC
AGAGCCACCCCCCCACCTCGTCTCCTCGCACCATGAGCTGGCTGTGCT
GTTTAGGACAGATCATGGCATCAGCAGTGGAGGCTTCTCAGCCACCTA
CCTGGCCTTCAATGCCACGGAGAACCCCTGTGGGCCCAGTGAGCTCTC
CTGCCAGGCAGGAGGGTGTAAGGGTGTGCAGTGGATGTGTGACATGTG
GAGAGACTGCACCGATGGCAGCGATGACAACTGCAGCGGCCCCTTGTT
CCCACCCCCAGAGCTGGCCTGTGAGCCTGTCCAGGTGGAGATGTGCCT
CGGTCTGAGCTACAACACCACAGCCTTCCCTAACATCTGGGTGGGCAT
GATCACCCAGGAGGAGGTGGTAGAGGTCCTCAGCGGTTACAAGAGCCT
GACAAGCCTGCCCTGCTACCAGCATTTCCGGAGGCTCCTGTGTGGGCT
GCTTGTGCCCCGTTGCACCCCACTAGGCAGTGTTCTGCCCCCTTGCCG
CTCTGTCTGCCAGGAAGCGGAGCACCAGTGCCAGTCTGGCCTGGCACT
ACTGGGCACCCCCTGGCCCTTCAACTGCAACAGGCTGCCAGAGGCAGC
TGACCTGGAAGCTTGTGCCCAGCCC

[symbol]

SEQ ID 2: Homo sapiens adiponectin receptor 1 (ADIPOR1)

[symbol]

TCTTCCCACAAAGGATCTGTGGTGGCAC
AGGGGAATGGGGCTCCTGCCAGTAACAGGGAAGCTGACACGGTGGAA
CTGGCTGAACTGGGACCCCTGCTAGAAGAGAAGGGCAAACGGGTAAT
CGCCAACCCACCCAAAGCTGAAGAAGAGCAAACATGCCCAGTGCCCC
AGGAAGAAGAGGAGGAGGTGCGGGTACTGACACTTCCCCTGCAAGCC
CACCACGCCATGGAGAAGATGGAAGAGTTTGTGTACAAGGTCTGGGA
GGGACGTTGGAGGGTCATCCCATATGATGTGCTCCCTGACTGGCTAA
AGGACAACGACTATCTGCTACATGGTCATAGACCTCCCATGCCCTCC
TTTCGGGCTTGCTTCAAGAGCATCTTCCGCATTCATACAGAAACTGG
CAACATCTGGACCCATCTGCTTGGTTTCGTGCTGTTTCTCTTTTTGG
GAATCTTGACCATGCTCAGACCAAATATGTACTTCATGGCCCCTCTA
CAGGAGAAGGTGGTTTTTGGGATGTTCTTTTTGGGTGCAGTGCTCTG
CCTCAGCTTCTCCTGGCTCTTTCACACCGTCTATTGTCATTCAGAGA
AAGTCTCTCGGACTTTTTCCAAACTGGACTATTCAGGGATTGCTCTT
CTAATTATGGGGAGCTTTGTCCCCTGGCTCTATTATTCCTTCTACTG
CTCCCCACAGCCACGGCTCATCTACCTCTCCATCGTCTGTGTCCTGG
GCATTTCTGCCATCATTGTGGCGCAGTGGGACCGGTTTGCCACTCCT
AAGCACCGGCAGACAAGAGCAGGCGTGTTCCTGGGACTTGGCTTGAG
TGGCGTCGTGCCCACCATGCACTTTACTATCGCTGAGGGCTTTGTCA
AGGCCACCACAGTGGGCCAGATGGGCTGGTTCTTCCTCATGGCTGTG
ATGTACATCACTGGAGCTGGCCTTTATGCTGCTCGAATTCCTGAGCG
CTTCTTTCCTGGAAAATTTGACATATGGTTCCAGTCTCATCAGATTT
TCCATGTCCTGGTGGTGGCAGCAGCCTTTGTCCACTTCTATGGAGTC
TCCAACCTTCAGGAATTCCGTTACGGCCTAGAAGGCGGCTGTACTGA
TGACACCCTTCTC

[symbol]

SEQ ID NO: 3 amino acid sequence of adiponectin receptor protein 1 [Homo sapiens] NCBI Reference Sequence: NP_001277558.1

| | |
|---|---|
| 1 | msshkgsvva qgngapasnr eadtvelael gplleekgkr vianppkaee eqtcpvpqee |
| 61 | eeevrvltlp lqahhamekm eefvykvweg rwrvipydvl pdwlkdndyl lhghrppmps |
| 121 | fracfksifr ihtetgniwt hllgfvlflf lgiltmlrpn myfmaplqek vvfgmfflga |
| 181 | vlclsfswlf htvychsekv srtfskldys giallimgsf vpwlyysfyc spqprliyls |
| 241 | ivcvlgisai ivaqwdrfat pkhrqtragv flglglsgvv ptmhftiaeg fvkattvgqm |
| 301 | gwfflmavmy itgaglyaar iperffpgkf diwfqshqif hvlvvaaafv hfygvsnlqe |
| 361 | frygleggct ddtll |

SEQ ID NO: 4 amino acid sequence of adiponectin receptor protein 1 [Mus musculus] NCBI Reference Sequence: NP_001292998.1

| | |
|---|---|
| 1 | msshkgsaga qgngapsgnr eadtvelael gplleekgkr aasspakaee dqacpvpqee |
| 61 | eeevrvltlp lqahhamekm eefvykyweg rwrvipydvl pdwlkdndyl lhghrppmps |
| 121 | fracfksifr ihtetgniwt hllgfvlflf lgiltmlrpn myfmaplqek vvfgmfflga |
| 181 | vlclsfswlf htvychsekv srtfskldys giallimgsf vpwlyysfyc spqprliyls |
| 241 | ivcvlgisai ivaqwdrfat pkhrqtragv flglglsgvv ptmhftiaeg fvkattvgqm |
| 301 | gwfflmavmy itgaglyaar iperffpgkf diwfqshqif hvlvvaaafv hfygvsnlqe |
| 361 | frygleggct ddsll |

SEQ ID NO: 5 amino acid sequence of membrane-type frizzled-related protein MFRP [Homo sapiens] GenBank: BAB39771.1

| | |
|---|---|
| 1 | mkdfsdvilc meatesskte fcnpafepes gppcpppvfp edasysvpap whgrrprglr |
| 61 | pdcrfswlcv lllsslllll lgllvaiila qlqaappsga shsplpaggl ttttttptit |
| 121 | tsqaagtpkg qqesgvspsp qstcggllsg prgffsspny pdpyppnthc vwhiqvatdh |
| 181 | aiqlkieals iesvasclfd rlelspepeg pllrvcgrvp pptlntnash llvvfvsdss |
| 241 | vegfgfhawy qamapgrgsc ahdefrcdql icllpdsvcd gfancadgsd etncsakfsg |
| 301 | cggnltglqg tfstpsylqq yphqllctwh isvpaghsie lqfhnfslea qdeckfdyve |
| 361 | vyetsssgaf sllgrfcgae ppphlvsshh elavlfrtdh gissggfsat ylafnatenp |
| 421 | cgpselscqa ggckgvqwmc dmwrdctdgs ddncsgplfp ppelacepvq vemclglsyn |
| 481 | ttafpniwvg mitqeevvev lsgyksltsl pcyqhfrrll cgllvprctp lgsvlppcrs |
| 541 | vcqeaehqcq sglallgtpw pfncnrlpea adleacaqp |

SEQ ID NO: 6 amino acid sequence of membrane frizzled-related protein isoform 2 [Mus musculus] (NCBI Reference Sequence: NP_001177243.1)

| | |
|---|---|
| 1 | mkdyddvilr peaselskte fcnpafdpea gpscpppalq rdvgsrlqap whaqrlrglq |
| 61 | pdchfswfci lllsglllll lgllvavila qlqatslprt tknplltrgl tpmgvipstt |
| 121 | pntttttttt tpartgqqea amspthqttc ggllpgpsgf fsspnypdly pplshcvwhi |
| 181 | qvaagqtiql kiqalsiesm ltclfdrlei iseptgpllr vcgktppatl ntntshlrvs |
| 241 | fvsdndvegs gfqawyqava pghwscahne fhcdlllclk rdsvcdgite cadgsdeanc |
| 301 | saktlgcggn ltglygvfst pnypqhyphq qlctwyievp vgygirlefh nfsleaqaec |
| 361 | kfdyvevyea snlgtfsflg rfcgaeppln vvssmhqlav ifktdlgiss ggflatyqai |
| 421 | nttekfcqsg gyrdlqwmcd lwkdcandsn dncsshlspq pdltcepvqv emclglsynt |
| 481 | tafpniwvgl atqtevtdil rgyksltslp cyqtfqrflc gllvprctsl gtilppcrsv |
| 541 | cqaaeqqcqs slallgtpwp fncnrlpvaa sleacsqp |

SEQ ID NO: 7 amino acid sequence of membrane frizzled-related protein isoform 1 [Mus musculus](NCBI Reference Sequence: NP_667337.1)

| | |
|---|---|
| 1 | mkdyddvilr peaselskte fcnpafdpea gpscpppalq rdvgsrlqap whaqrlrglq |
| 61 | pdchfswfci lllsglllll lgllvavila qlqatslprt tknplltrgl tpmgvipstt |
| 121 | pntttttttt tpartgqqea amspthqttc ggllpgpsgf fsspnypdly pplshcvwhi |
| 181 | qvaagqtiql kiqalsiesm ltclfdrlei iseptgpllr vcgktppatl ntntshlrvs |
| 241 | fvsdndvegs gfqawyqava pghwscahne fhcdlllclk rdsvcdgite cadgsdeanc |
| 301 | saktlgcggn ltglygvfst pnypqhyphq qlctwyievp vgygirlefh nfsleaqaec |
| 361 | kfdyvevyea snlgtfsflg rfcgaeppin vvssmhqlav ifktdlgiss ggflatyqai |
| 421 | nttesgcpwa efcqsggyrd lqwmcdlwkd candsndncs shlspqpdlt cepvqvemcl |
| 481 | glsynttafp niwvglatqt evtdilrgyk sltslpcyqt fqrflcgllv prctslgtil |
| 541 | ppersvcqaa eqqcqsslal lgtpwpfncn rlpvaaslea csqp |

SEQ ID 8—Homo sapiens membrane frizzled-related protein (MFRP)

AAGGACTTCTCAGATGTCATCCTCTGCATGGAGGCAACAGAATCGAGCAAGACC
GAGTTCTGCAATCCTGCCTTCGAGCCTGAGTCTGGGCCACCCTGCCCTCCCCCAG
TTTTCCCAGAGGATGCCAGCTACAGCGTCCCAGCTCCCTGGCATGGTCGGCGTCC
TCGAGGGCTACGGCCAGACTGCCGCTTCTCCTGGCTCTGTGTCCTCCTGCTCTCCA
GCCTGCTCCTCCTGCTGCTTGGGCTGCTGGTGGCCATCATCCTGGCCCAGCTGCA
GGCTGCACCCCCATCTGGGGCGTCCCATAGCCCACTGCCTGCCGGAGGCCTTACC
ACGACCACCACCACCCCCACCATCACCACCTCTCAGGCAGCTGGGACCCCTAAA
GGGCAGCAGGAGTCAGGCGTGAGCCCCTCCCCACAGTCCACCTGTGGAGGCCTC
CTCTCTGGCCCAAGGGGCTTCTTCAGCAGCCCTAACTACCCAGACCCTTACCCCC
CCAACACCCACTGCGTGTGGCATATCCAGGTGGCCACAGACCATGCAATACAGC
TCAAGATCGAAGCCCTCAGCATAGAGAGTGTGGCCTCTTGCCTTTTTGATCGCTT
GGAACTCTCCCCTGAGCCTGAAGGCCCCCTCCTCAGGGTTTGTGGAAGGGTGCCT
CCCCCCACGCTCAACACCAATGCCAGCCACCTCCTGGTGGTCTTCGTCTCTGACA
GCAGTGTGGAAGGATTTGGTTTCCATGCCTGGTACCAGGCTATGGCCCCTGGGCG
CGGGAGCTGTGCCCATGATGAGTTCCGCTGTGACCAGCTCATCTGCCTGCTACCT
GACTCAGTGTGTGATGGTTTTGCCAACTGTGCTGACGGCAGTGATGAGACCAATT
GCAGTGCCAAGTTCTCGGGGTGTGGGGGGAATCTGACTGGCCTCCAGGGCACTTT
CTCTACTCCCAGCTACCTGCAGCAGTACCCTCACCAACTGCTCTGCACCTGGCAT
ATCTCGGTGCCTGCCGGACACAGCATAGAACTACAGTTCCACAACTTCAGCCTGG
AGGCTCAGGACGAGTGCAAGTTTGACTACGTGGAGGTGTATGAGACCAGCAGCT
CAGGGGCCTTCAGCCTCCTGGGCAGGTTCTGTGGAGCAGAGCCACCCCCCCACCT
CGTCTCCTCGCACCATGAGCTGGCTGTGCTGTTTAGGACAGATCATGGCATCAGC
AGTGGAGGCTTCTCAGCCACCTACCTGGCCTTCAATGCCACGGAGAACCCCTGTG
GGCCCAGTGAGCTCTCCTGCCAGGCAGGAGGGTGTAAGGGTGTGCAGTGGATGT
GTGACATGTGGAGAGACTGCACCGATGGCAGCGATGACAACTGCAGCGGCCCCT
TGTTCCCACCCCCAGAGCTGGCCTGTGAGCCTGTCCAGGTGGAGATGTGCCTCGG
TCTGAGCTACAACACCACAGCCTTCCCTAACATCTGGGTGGGCATGATCACCCAG
GAGGAGGTGGTAGAGGTCCTCAGCGGTTACAAGAGCCTGACAAGCCTGCCCTGC
TACCAGCATTTCCGGAGGCTCCTGTGTGGGCTGCTTGTGCCCCGTTGCACCCCAC
TAGGCAGTGTTCTGCCCCCTTGCCGCTCTGTCTGCCAGGAAGCGGAGCACCAGTG
CCAGTCTGGCCTGGCACTACTGGGCACCCCCTGGCCCTTCAACTGCAACAGGCTG
CCAGAGGCAGCTGACCTGGAAGCTTGTGCCCAGCCC
SEQ ID 9: Homo sapiens adiponectin receptor 1
(ADIPOR1)
TCTTCCCACAAAGGATCTGTGGTGGCACAGGGGAATGGGGCTCCTGCCAGTAAC
AGGGAAGCTGACACGGTGGAACTGGCTGAACTGGGACCCCTGCTAGAAGAGAAG
GGCAAACGGGTAATCGCCAACCCACCCAAAGCTGAAGAAGAGCAAACATGCCC
AGTGCCCCAGGAAGAAGAGGAGGAGGTGCGGGTACTGACACTTCCCCTGCAAGC
CCACCACGCCATGGAGAAGATGGAAGAGTTTGTGTACAAGGTCTGGGAGGGACG
TTGGAGGGTCATCCCATATGATGTGCTCCCTGACTGGCTAAAGGACAACGACTAT
CTGCTACATGGTCATAGACCTCCCATGCCCTCCTTTCGGGCTTGCTTCAAGAGCAT
CTTCCGCATTCATACAGAAACTGGCAACATCTGGACCCATCTGCTTGGTTTCGTG
CTGTTTCTCTTTTTGGGAATCTTGACCATGCTCAGACCAAATATGTACTTCATGGC
CCCTCTACAGGAGAAGGTGGTTTTTGGGATGTTCTTTTTGGGTGCAGTGCTCTGCC
TCAGCTTCTCCTGGCTCTTTCACACCGTCTATTGTCATTCAGAGAAAGTCTCTCGG
ACTTTTTCCAAACTGGACTATTCAGGGATTGCTCTTCTAATTATGGGGAGCTTTGT
CCCCTGGCTCTATTATTCCTTCTACTGCTCCCCACAGCCACGGCTCATCTACCTCT
CCATCGTCTGTGTCCTGGGCATTTCTGCCATCATTGTGGCGCAGTGGGACCGGTTT
GCCACTCCTAAGCACCGGCAGACAAGAGCAGGCGTGTTCCTGGGACTTGGCTTG
AGTGGCGTCGTGCCCACCATGCACTTTACTATCGCTGAGGGCTTTGTCAAGGCCA
CCACAGTGGGCCAGATGGGCTGGTTCTTCCTCATGGCTGTGATGTACATCACTGG
AGCTGGCCTTTATGCTGCTCGAATTCCTGAGCGCTTCTTTCCTGGAAAATTTGACA
TATGGTTCCAGTCTCATCAGATTTTCCATGTCCTGGTGGTGGCAGCAGCCTTTGTC
CACTTCTATGGAGTCTCCAACCTTCAGGAATTCCGTTACGGCCTAGAAGGCGGCT
GTACTGATGACACCCTTCTC
SEQ ID 10: EX-W01610-Lv03 Homo sapiens membrane
frizzled-related protein (MFRP)
AACCCAGCTTTCTTGTACAAAGTGGTTGATCGCGTGCATGCGACGTCATAGCTCTCTCCCAA
TTCTCGACCTCGAGACAAATGGCAGTATTCATCCACAATTTTAAAAGAAAAGGGGGGATTGG
GGGGTACAGTGCAGGGGAAAGAATAGTAGACATAATAGCAACAGACATACAAACTAAAGAAT
TACAAAAACAAATTACAAAAATTCAAAATTTTCGGGTTTATTACAGGGACAGCAGAGATCCA
CTTTGGCCGCGGCTCGAGGGGGTTGGGGTTGCGCCTTTTCCAAGGCAGCCCTGGGTTTGCGC
AGGGACGCGGCTGCTCTGGGCGTGGTTCCGGGAAACGCAGCGGCGCCGACCCTGGGTCTCGC
ACATTCTTCACGTCCGTTCGCAGCGTCACCCGGATCTTCGCCGCTACCCTTGTGGGCCCCCC
GGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCA
ATGGCAGCGCGCCGACCGCGATGGGCTGTGGCCAATAGCGGCTGCTCAGCAGGGCGCGCCGA
GAGCAGCGGCCGGGAAGGGGCGGTGCGGGAGGCGGGGTGTGGGGCGGTAGTGTGGGCCCTGT
TCCTGCCCGCGCGGTGTTCCGCATTCTGCAAGCCTCCGGAGCGCACGTCGGCAGTCGGCTCC
CTCGTTGACCGAATCACCGACCTCTCTCCCCAGGGGGATCCACCGGAGCTTACCATGACCGA
GTACAAGCCCACGGTGCGCCTCGCCACCCGCGACGACGTCCCCAGGGCGGTACGCACCCTCG
CCGCCGCGTTCGCCGACTACCCCGCCACGCGCCACACCGTCGATCCGGACCGCCACATCGAG
CGGGTCACCGAGCTGCAAGAACTCTTCCTCACGCGCGTCGGGCTCGACATCGGCAAGGTGTG
GGTCGCGGACGACGGCGCCGCGGTGGCGGTCTGGACCACGCCGGAGAGCGTCGAAGCGGGGG
CGGTGTTCGCCGAGATCGGCCCGCGCATGGCCGAGTTGAGCGGTTCCCGGCTGGCCGCGCAG
CAACAGATGGAAGGCCTCCTGGCGCCGCACCGGCCCAAGGAGCCCGCGTGGTTCCTGGCCAC
CGTCGGCGTCTCGCCCGACCACCAGGGCAAGGGTCTGGGCAGCGCCGTCGTGCTCCCCGGAG
TGGAGGCGGCCGAGCGCGCCGGGGTGCCCGCCTTCCTGGAGACCTCCGCGCCCCGCAACCTC
CCCTTCTACGAGCGGCTCGGCTTCACCGTCACCGCCGACGTCGAGGTGCCCGAAGGACCGCG
CACCTGGTGCATGACCCGCAAGCCCGGTGCCTGACGCCCGCCCCACGACCCGCAGCGCCCGA
CCGAAAGGAGCGCACGACCCCATGCATCGGTACCTTTAAGACCAATGACTTACAAGGCAGCT
GTAGATCTTAGCCACTTTTTAAAAGAAAAGGGGGGACTGGAAGGGCTAATTCACTCCCAACG
AAGACAAGATCTGCTTTTTGCTTGTACTGGGTCTCTCTGGTTAGACCAGATCTGAGCCTGGG
AGCTCTCTGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGACCCTTTTA
GTCAGTGTGGAAAATCTCTAGCAGTAGTAGTTCATGTCATCTTATTATTCAGTATTTATAAC
TTGCAAAGAAATGAATATCAGAGAGTGAGAGGAACTTGTTTATTGCAGCTTATAATGGTTAC
AAATAAAGCAATAGCATCACAAATTTCACAAATAAAGCATTTTTTTCACTGCATTCTAGTTG
TGGTTTGTCCAAACTCATCAATGTATCTTATCATGTCTGGCTCTAGCTATCCCGCCCCTAAC
TCCGCCCATCCCGCCCCTAACTCCGCCCAGTTCCGCCCATTCTCCGCCCCATGGCTGACTAA
TTTTTTTTATTTATGCAGAGGCCGAGGCCGCCTCGGCCTCTGAGCTATTCCAGAAGTAGTGA
GGAGGCTTTTTTGGAGGCCTAGGGACGTACCCAATTCGCCCTATAGTGAGTCGTATTACGCG
CGCTCACTGGCCGTCGTTTTACAACGTCGTGACTGGGAAAACCCTGGCGTTACCCAACTTAA
TCGCCTTGCAGCACATCCCCCTTTCGCCAGCTGGCGTAATAGCGAAGAGGCCCGCACCGATC
GCCCTTCCCAACAGTTGCGCAGCCTGAATGGCGAATGGGACGCGCCCTGTAGCGGCGCATTA
AGCGCGGCGGGTGTGGTGGTTACGCGCAGCGTGACCGCTACACTTGCCAGCGCCCTAGCGCC
CGCTCCTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTCGCCCTTT
GACGTTGGAGTCCACGTTCTTTAATAGTGGACTCTTGTTCCAAACTGGAACAACACTCAACC
CTATCTCGGTCTATTCTTTTGATTTATAAGGGATTTTGCCGATTTCGGCCTATTGGTTAAAA
AATGAGCTGATTTAACAAAAATTTAACGCGAATTTTAACAAAATATTAACGCTTACAATTTA
GGTGGCACTTTTCGGGGAAATGTGCGCGGAACCCCTATTTGTTTATTTTTCTAAATACATTC
AAATATGTATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTTTGCCTT
CCTGTTTTTGCTCACCCAGAAACGCTGGTGAAAGTAAAAGATGCTGAAGATCAGTTGGGTGC
ACGAGTGGGTTACATCGAACTGGATCTCAACAGCGGTAAGATCCTTGAGAGTTTTCGCCCCG
AAGAACGTTTTCCAATGATGAGCACTTTTAAAGTTCTGCTATGTGGCGCGGTATTATCCCGT
ATTGACGCCGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATTATGCAGTG
CTGCCATAACCATGAGTGATAACACTGCGGCCAACTTACTTCTGACAACGATCGGAGGACCG
AAGGAGCTAACCGCTTTTTTGCACAACATGGGGGATCATGTAACTCGCCTTGATCGTTGGGA
ACCGGAGCTGAATGAAGCCATACCAAACGACGAGCGTGACACCACGATGCCTGTAGCAATGG
CAACAACGTTGCGCAAACTATTAACTGGCGAACTACTTACTCTAGCTTCCCGGCAACAATTA
ATAGACTGGATGGAGGCGGATAAAGTTGCAGGACCACTTCTGCGCTCGGCCCTTCCGGCTGG
CTGGTTTATTGCTGATAAATCTGGAGCCGGTGAGCGTGGGTCTCGCGGTATCATTGCAGCAC
TGGGGCCAGATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCATTGGTAACT
GTCAGACCAAGTTTACTCATATATACTTTAGATTGATTTAAAACTTCATTTTTAATTTAAAA
GGATCTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCAAAT
ACTGTTCTTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGA
GCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGGGGGGCG
GAGCCTATGGAAAAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGTATTACCGCCTTT
GAGTGAGCTGATACCGCTCGCCGCAGCCGAACGACCGAGCGCAGCGAGTCAGTGAGCGAGGA
AGCGGAAGAGCGCCCTTTGAGTGAGCTGATACCGCTCGCCGCAGCCGAACGACCGAGCGCAG
CGAGTCAGTGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCAGTGAGCGC
AACGCAAAATGTGAGTTAGCTCACTCATTAGGCACCCCAGGCTTTACACTTTATGCTTCCGG
CTCGTATGTTGTGTGGAATTGTGAGCGGATAACAATTTCACACAGGAAACAGCTATGACCAT
GATTACGCCAAGCGCGCAATTAACCCTCACTAAAGGGAACAAAAGCTGGAGCTGCAAGCTTA
ATGTAGTCTTATGCAATACTCTTGTAGTCTTGCAACATGGTAACGATGAGTTAGCAACATGC
CTTACAAGGAGAGAAAAAGCACCGTGCATGCCGATTGGTGGAAGTAAGGTGGTACGATCGTG
CCTTATTAGGAAGGCAACAGACGGGTCTGACATGGATTGGACGAACCACTGAATTGCCGCAT
TGCAGAGATATTGTATTTAAGTGCCTAGCTCGATACATAAACGGGTCTCTCTGGTTAGACCA
GATCTGAGCCTGGGAGCTCTCTGGCTAACTAGGGAACCCACTGCTTAAGCCTCAATAAAGCT
TGCCTTGAGTGCTTCAAGTAGTGTGTGCCCGTCTGTTGTGTGACTCTGGTAACTAGAGATCC
CTCAGACCCTTTTAGTCAGTGTGGAAAATCTCTAGCAGTGGCGCCCGAACAGGGACTTGAAA
GCGAAAGGGAAACCAGAGGAGCTCTCTCGACGCAGGACTCGGCTTGCTGAAGCGCGCACGGC
AAGAGGCGAGGGGCGGCGACTGGTGAGTACGCCAAAAATTTTGACTAGCGGAGGCTAGAAGG
AGAGAGATGGGTGCGAGAGCGTCAGTATTAAGCGGGGGAGAATTAGATCGCGATGGGAAAAA
ATTCGGTTAAGGCCAGGGGGAAAGAAAAAATATAAATTAAAACATATAGTATGGGCAAGCAG
GGAGCTAGAACGATTCGCAGTTAATCCTGGCCTGTTAGAAACATCAGAAGGCTGTAGACAAA
TACTGGGACAGCTACAACCATCCCTTCAGACAGGATCAGAAGAACTTAGATCATTATATAAT
ACAGTAGCAACCCTCTATTGTGTGCATCAAAGGATAGAGATAAAAGACACCAAGGAAGCTTT
AGACAAGATAGAGGAAGAGCAAAACAAAAGTAAGACCACCGCACAGCAAGCGGCCGCTGATC
TTCAGACCTGGAGGAGGAGATATGAGGGACAATTGGAGAAGTGAATTATATAAATATAAAGT
AGTAAAAATTGAACCATTAGGAGTAGCACCCACCAAGGCAAAGAGAAGAGTGGTGCAGAGAG
AAAAAAGAGCAGTGGGAATAGGAGCTTTGTTCCTTGGGTTCTTGGGAGCAGCAGGAAGCACT
ATGGGCGCAGCGTCAATGACGCTGACGGTACAGGCCAGACAATTATTGTCTGGTATAGTGCA
GCAGCAGAACAATTTGCTGAGGGCTATTGAGGCGCAACAGCATCTGTTGCAACTCACAGTCT
GGGGCATCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGGAATGC
TAGTTGGAGTAATAAATCTCTGGAACAGATTTGGAATCACACGACCTGGATGGAGTGGGACA
GAGAAATTAACAATTACACAAGCTTAATACACTCCTTAATTGAAGAATCGCAAAACCAGCAA
GAAAAGAATGAACAAGAATTATTGGAATTAGATAAATGGGCAAGTTTGTGGAATTGGTTTAA
CATAACAAATTGGCTGTGGTATATAAAATTATTCATAATGATAGTAGGAGGCTTGGTAGGTT
TAAGAATAGTTTTTGCTGTACTTTCTATAGTGAATAGAGTTAGGCAGGGATATTCACCATTA
TCGTTTCAGACCCACCTCCCAACCCCGAGGGGACCCGACAGGCCCGAAGGAATAGAAGAAGA
AGGTGGAGAGAGAGACAGAGACAGATCCATTCGATTAGTGAACGGATCTCGACGGTATCGAT
CACGAGACTAGCCTCGAGCGGCCGCCCCCTTCACCGAGGGCCTATTTCCCATGATTCCTTCA
TATTTGCATATACGATACAAGGCTGTTAGAGAGATAATTGGAATTAATTTGACTGTAAACAC
AAAGATATTAGTACAAAATACGTGACGTAGAAAGTAATAATTTCTTGGGTAGTTTGCAGTTT
TAAAATTATGTTTTAAAATGGACTATCATATGCTTACCGTAACTTGAAAGTATTTCGATTTC
TTGGCTTTATATATCTTGTGGAAAGGACGAAACACCGGTATCTATACATTGAATCAATATTG
GCAATTAGCCATATTAGTCATTGGTTATATAGCATAAATCAATATTGGCTATTGGCCATTGC
ATACGTTGTATCTATATCATAATATGTACATTTATATTGGCTCATGTCCAATATGACCGCCA
TGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAGGGACT
TTCCATTGACGTCAATGGGTGGAGTATTTACGGTAAACTGCCCACTTGGCAGTACATCAAGT
GTATCATATGCCAAGTCCGCCCCCTATTGACGTCAATGACGGTAAATGGCCCGCCTGGCATT
ATGCCCAGTACATGACCTTACGGGACTTTCCTACTTGGCAGTACATCTACGTATTAGTCATC
GCTATTACCATGGTGATGCGGTTTTGGCAGTACATCAATGGGCGTGGATAGCGGTTTGACTC
ACGGGGATTTCCAAGTCTCCACCCCATTGACGTCAATGGGAGTTTGTTTTGGCACCAAAATC
AACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGTCAGATCGCCTGGAGACG
CCATCCACGCTGTTTTGACCTCCATAGAAGATTCTAGAACCATGGTGAGCAAGGGCGAGGAG
CTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCT
GCACCACCGGCAAGCTGCCCGTGCCCTGGCCCACCCTCGTGACCACCCTGACCTACGGCGTG
CAGTGCTTCAGCCGCTACCCCGACCACATGAAGCAGCACGACTTCTTCAAGTCCGCCATGCC
CGAAGGCTACGTCCAGGAGCGCACCATCTTCTTCAAGGACGACGGCAACTACAAGACCCGCG
CCGAGGTGAAGTTCGAGGGCGACACCCTGGTGAACCGCATCGAGCTGAAGGGCATCGACTTC
AAGGAGGACGGCAACATCCTGGGGCACAAGCTGGAGTACAACTACAACAGCCACAACGTCTA
TATCATGGCCGACAAGCAGAAGAACGGCATCAAGGTGAACTTCAAGATCCGCCACAACATCG
AGGACGGCAGCGTGCAGCTCGCCGACCACTACCAGCAGAACACCCCCATCGGCGACGGCCCC
GTGCTGCTGCCCGACAACCACTACCTGAGCACCCAGTCCGCCCTGAGCAAAGACCCCAACGA
GAAGCGCGATCACATGGTCCTGCTGGAGTTCGTGACCGCCGCCGGGATCACTCTCGGCATGG

[chemical structure]

SEQ ID 11: EX-Z1681-Lv111 Homo sapiens adiponectin receptor 1
(ADIPOR1)
AACCCAGCTTTCTTGTACAAAGTGGTTGATCGCGTGCATGCGACGTCATAGCTCTCTCCC
AATTCTCGACCTCGAGACAAATGGCAGTATTCATCCACAATTTTAAAAGAAAAGGGGGGA
TTGGGGGGTACAGTGCAGGGGAAAGAATAGTAGACATAATAGCAACAGACATACAAACTA
AAGAATTACAAAAACAAATTACAAAAATTCAAAATTTTCGGGTTTATTACAGGGACAGCA
GAGATCCACTTTGGCCGCGGCTCGAGGGGGTTGGGGTTGCGCCTTTTCCAAGGCAGCCCT
GGGTTTGCGCAGGGACGCGGCTGCTCTGGGCGTGGTTCCGGGAAACGCAGCGGCGCCGAC
CCTGGGTCTCGCACATTCTTCACGTCCGTTCGCAGCGTCACCCGGATCTTCGCCGCTACC
CTTGTGGGCCCCCCGGCGACGCTTCCTGCTCCGCCCCTAAGTCGGGAAGGTTCCTTGCGG
TTCGCGGCGTGCCGGACGTGACAAACGGAAGCCGCACGTCTCACTAGTACCCTCGCAGAC
GGACAGCGCCAGGGAGCAATGGCAGCGCGCCGACCGCGATGGGCTGTGGCCAATAGCGGC
TGCTCAGCAGGGCGCGCCGAGAGCAGCGGCCGGGAAGGGGCGGTGCGGGAGGCGGGGTGT
GGGGCGGTAGTGTGGGCCCTGTTCCTGCCCGCGCGGTGTTCCGCATTCTGCAAGCCTCCG
GAGCGCACGTCGGCAGTCGGCTCCCTCGTTGACCGAATCACCGACCTCTCTCCCCAGGGG
GATCCACCGGAGCTTACCATGACCGAGTACAAGCCCACGGTGCGCCTCGCCACCCGCGAC
GACGTCCCCAGGGCGGTACGCACCCTCGCCGCCGCGTTCGCCGACTACCCCGCCACGCGC
CACACCGTCGATCCGGACCGCCACATCGAGCGGGTCACCGAGCTGCAAGAACTCTTCCTC
ACGCGCGTCGGGCTCGACATCGGCAAGGTGTGGGTCGCGGACGACGGCGCCGCGGTGGCG
GTCTGGACCACGCCGGAGAGCGTCGAAGCGGGGGCGGTGTTCGCCGAGATCGGCCCGCGC
ATGGCCGAGTTGAGCGGTTCCCGGCTGGCCGCGCAGCAACAGATGGAAGGCCTCCTGGCG
CCGCACCGGCCCAAGGAGCCCGCGTGGTTCCTGGCCACCGTCGGCGTCTCGCCCGACCAC
CAGGGCAAGGGTCTGGGCAGCGCCGTCGTGCTCCCCGGAGTGGAGGCGGCCGAGCGCGCC
GGGGTGCCCGCCTTCCTGGAGACCTCCGCGCCCCGCAACCTCCCCTTCTACGAGCGGCTC
GGCTTCACCGTCACCGCCGACGTCGAGGTGCCCGAAGGACCGCGCACCTGGTGCATGACC
CGCAAGCCCGGTGCCTGACGCCCGCCCCACGACCCGCAGCGCCCGACCGAAAGGAGCGCA
CGACCCCATGCATCGGTACCTTTAAGACCAATGACTTACAAGGCAGCTGTAGATCTTAGC
CACTTTTTAAAAGAAAAGGGGGGACTGGAAGGGCTAATTCACTCCCAACGAAGACAAGAT
CTGCTTTTTGCTTGTACTGGGTCTCTCTGGTTAGACCAGATCTGAGCCTGGGAGCTCTCT
GGCTAACTAGGGAACCCACTGCTTAAGCCTCAATAAAGCTTGCCTTGAGTGCTTCAAGTA
GTGTGTGCCCGTCTGTTGTGTGACTCTGGTAACTAGAGATCCCTCAGACCCTTTTAGTCA
GTGTGGAAAATCTCTAGCAGTAGTAGTTCATGTCATCTTATTATTCAGTATTTATAACTT
GCAAAGAAATGAATATCAGAGAGTGAGAGGAACTTGTTTATTGCAGCTTATAATGGTTAC
AAATAAAGCAATAGCATCACAAATTTCACAAATAAAGCATTTTTTTCACTGCATTCTAGT
TGTGGTTTGTCCAAACTCATCAATGTATCTTATCATGTCTGGCTCTAGCTATCCCGCCCC
TAACTCCGCCCATCCCGCCCCTAACTCCGCCCAGTTCCGCCCATTCTCCGCCCCATGGCT
GACTAATTTTTTTTATTTATGCAGAGGCCGAGGCCGCCTCGGCCTCTGAGCTATTCCAGA
AGTAGTGAGGAGGCTTTTTTGGAGGCCTAGGGACGTACCCAATTCGCCCTATAGTGAGTC
GTATTACGCGCGCTCACTGGCCGTCGTTTTACAACGTCGTGACTGGGAAAACCCTGGCGT
TACCCAACTTAATCGCCTTGCAGCACATCCCCCTTTCGCCAGCTGGCGTAATAGCGAAGA
GGCCCGCACCGATCGCCCTTCCCAACAGTTGCGCAGCCTGAATGGCGAATGGGACGCGCC
CTGTAGCGGCGCATTAAGCGCGGCGGGTGTGGTGGTTACGCGCAGCGTGACCGCTACACT
TGCCAGCGCCCTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTGGGCCATCGCC
CTGATAGACGGTTTTTCGCCCTTTGACGTTGGAGTCCACGTTCTTTAATAGTGGACTCTT
GTTCCAAACTGGAACAACACTCAACCCTATCTCGGTCTATTCTTTTGATTTATAAGGGAT
TTTGCCGATTTCGGCCTATTGGTTAAAAAATGAGCTGATTTAACAAAAATTTAACGCGAA
TTTTAACAAAATATTAACGCTTACAATTTAGGTGGCACTTTTCGGGGAAATGTGCGCGGA
ACCCCTATTTGTTTATTTTTCTAAATACATTCAAATATGTATCCGCTCATGAGACAATAA
CCCTGATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAATGATG
AGCACTTTTAAAGTTCTGCTATGTGGCGCGGTATTATCCCGTATTGACGCCGGGCAAGAG
CAACTCGGTCGCCGCATACACTATTCTCAGAATGACTTGGTTGAGTACTCACCAGTCACA
GAAAAGCATCTTACGGATGGCATGACAGTAAGAGAATTATGCAGTGCTGCCATAACCATG
AGTGATAACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGGAGCTG
AATGAAGCCATACCAAACGACGAGCGTGACACCACGATGCCTGTAGCAATGGCAACAACG
TTGCGCAAACTATTAACTGGCGAACTACTTACTCTAGCTTCCCGGCAACAATTAATAGAC
TGGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACTG
GGGCCAGATGGTAAGCCCTCCCGTATCGTAGTTATCTACACGACGGGGAGTCAGGCAACT
ATGGATGAACGAAATAGACAGATCGCTGAGATAGGTGCCTCACTGATTAAGCATTGGTAA
CTGTCAGACCAAGTTTACTCATATATACTTTAGATTGATTTAAAACTTCATTTTTAATTT
AAAAGGATCTAGGTGAAGATCCTTTTTGATAATCTCATGACCAAAATCCCTTAACGTGAG
TTTTCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGAGCG
CAGATACCAAATACTGTTCTTCTAGTGTAGCCGTAGTTAGGCCACCACTTCAAGAACTCT
GTAGCACCGCCTACATACCTCGCTCTGCTAATCCTGTTACCAGTGGCTGCTGCCAGTGGC
GATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAA
CTGAGATACCTACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGAGCTTCCAGGG
GGAAACGCCTGGTATCTTTATAGTCCTGTCGGGTTTCGCCACCTCTGACTTGAGCGTCGA
TTTTTGTGATGCTCGTCAGGGGGGCGGAGCCTATGGAAAAACGCCAGCAACGCGGCCTTT
TTACGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAGAGCGCCCTTTGAGTGAGCTG
ATACCGCTCGCCGCAGCCGAACGACCGAGCGCAGCGAGTCAGTGAGCGAGGAAGCGGAAG
AGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAAAATGTGAGTTAGCTC
ACTCATTAGGCACCCCAGGCTTTACACTTTATGCTTCCGGCTCGTATGTTGTGTGGAATT
GTGAGCGGATAACAATTTCACACAGGAAACAGCTATGACCATGATTACGCCAAGCGCGCA
ATTAACCCTCACTAAAGGGAACAAAAGCTGGAGCTGCAAGCTTAATGTAGTCTTATGCAA
TACTCTTGTAGTCTTGCAACATGGTAACGATGAGTTAGCAACATGCCTTACAAGGAGAGA
AAAAGCACCGTGCATGCCGATTGGTGGAAGTAAGGTGGTACGATCGTGCCTTATTAGGAA
GGCAACAGACGGGTCTGACATGGATTGGACGAACCACTGAATTGCCGCATTGCAGAGATA
TTGTATTTAAGTGCCTAGCTCGATACATAAACGGGTCTCTCTGGTTAGACCAGATCTGAG
CCTGGGAGCTCTCTGGCTAACTAGGGAACCCACTGCTTAAGCCTCAATAAAGCTTGCCTT
GAGTGCTTCAAGTAGTGTGTGCCCGTCTGTTGTGTGACTCTGGTAACTAGAGATCCCTCA
GACCCTTTTAGTCAGTGTGGAAAATCTCTAGCAGTGGCGCCCGAACAGGGACTTGAAAGC
GAAAGGGAAACCAGAGGAGCTCTCTCGACGCAGGACTCGGCTTGCTGAAGCGCGCACGGC
AAGAGGCGAGGGGCGGCGACTGGTGAGTACGCCAAAAATTTTGACTAGCGGAGGCTAGAA
GGAGAGAGATGGGTGCGAGAGCGTCAGTATTAAGCGGGGGAGAATTAGATCGCGATGGGA
AAAAATTCGGTTAAGGCCAGGGGGAAAGAAAAAATATAAATTAAAACATATAGTATGGGC
AAGCAGGGAGCTAGAACGATTCGCAGTTAATCCTGGCCTGTTAGAAACATCAGAAGGCTG
TAGACAAATACTGGGACAGCTACAACCATCCCTTCAGACAGGATCAGAAGAACTTAGATC
ATTATATAATACAGTAGCAACCCTCTATTGTGTGCATCAAAGGATAGAGATAAAAGACAC
CAAGGAAGCTTTAGACAAGATAGAGGAAGAGCAAAACAAAAGTAAGACCACCGCACAGCA
AGCGGCCGCTGATCTTCAGACCTGGAGGAGGAGATATGAGGGACAATTGGAGAAGTGAAT
TATATAAATATAAAGTAGTAAAAATTGAACCATTAGGAGTAGCACCCACCAAGGCAAAGA
GAAGAGTGGTGCAGAGAGAAAAAAGAGCAGTGGGAATAGGAGCTTTGTTCCTTGGGTTCT
TGGGAGCAGCAGGAAGCACTATGGGCGCAGCGTCAATGACGCTGACGGTACAGGCCAGAC
AATTATTGTCTGGTATAGTGCAGCAGCAGAACAATTTGCTGAGGGCTATTGAGGCGCAAC
AGCATCTGTTGCAACTCACAGTCTGGGGCATCAAGCAGCTCCAGGCAAGAATCCTGGCTG
TGGAAAGATACCTAAAGGATCAACAGCTCCTGGGGATTTGGGGTTGCTCTGGAAAACTCA
TTTGCACCACTGCTGTGCCTTGGAATGCTAGTTGGAGTAATAAATCTCTGGAACAGATTT
GGAATCACACGACCTGGATGGAGTGGGACAGAGAAATTAACAATTACACAAGCTTAATAC
ACTCCTTAATTGAAGAATCGCAAAACCAGCAAGAAAAGAATGAACAAGAATTATTGGAAT
TAGATAAATGGGCAAGTTTGTGGAATTGGTTTAACATAACAAATTGGCTGTGGTATATAA
AATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAA
CCCCGAGGGGACCCGACAGGCCCGAAGGAATAGAAGAAGAAGGTGGAGAGAGAGACAGAG
ACAGATCCATTCGATTAGTGAACGGATCTCGACGGTATCGATCACGAGACTAGCCTCGAG
CGGCCGCCCCCTTCACCGAGGGCCTATTTCCCATGATTCCTTCATATTTGCATATACGAT
ACAAGGCTGTTAGAGAGATAATTGGAATTAATTTGACTGTAAACACAAAGATATTAGTAC
AAAATACGTGACGTAGAAAGTAATAATTTCTTGGGTAGTTTGCAGTTTTAAAATTATGTT
TTAAAATGGACTATCATATGCTTACCGTAACTTGAAAGTATTTCGATTTCTTGGCTTTAT
ATATCTTGTGGAAAGGACGAAACACCGGTATCTATACATTGAATCAATATTGGCAATTAG
CCATATTAGTCATTGGTTATATAGCATAAATCAATATTGGCTATTGGCCATTGCATACGT
TGTATCTATATCATAATATGTACATTTATATTGGCTCATGTCCAATATGACCGCCATGTT
GACATTGATTATTGACTAGTTATTAATAGTAATCAATTACGGGGTCATTAGTTCATAGCC
CATATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGTACATC
AAGTGTATCATATGCCAAGTCCGCCCCCTATTGACGTCAATGACGGTAAATGGCCCGCCT
GGCATTATGCCCAGTACATGACCTTACGGGACTTTCCTACTTGGCAGTACATCTACGTAT
TAGTCATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTTGTTTT
GGCACCAAAATCAACGGGACTTTCCAAAATGTCGTAACAACTCCGCCCCATTGACGCAAA
TGGGCGGTAGGCGTGTACGGTGGGAGGTCTATATAAGCAGAGCTCGTTTAGTGAACCGTC
AGATCGCCTGGAGACGCCATCCACGCTGTTTTGACCTCCATAGAAGATTCTAGAACCATG
GTGAGCAAGGGCGAGGAGGATAACATGGCCATCATCAAGGAGTTCATGCGCTTCAAGGTG
CACATGGAGGGCTCCGTGAACGGCCACGAGTTCGAGATCGAGGGCGAGGGCGAGGGCCGC
CCCTACGAGGGCACCCAGACCGCCAAGCTGAAGGTGACCAAGGGTGGCCCCCTGCCCTTC
GCCTGGGACATCCTGTCCCCTCAGTTCATGTACGGCTCCAAGGCCTACGTGAAGCACCCC
GCCGACATCCCCGACTACTTGAAGCTGTCCTTCCCCGAGGGCTTCAAGTGGGAGCGCGTG
ATGAACTTCGAGGACGGCGGCGTGGTGACCGTGACCCAGGACTCCTCCCTGCAGGACGGC
GAGTTCATCTACAAGGTGAAGCTGCGCGGCACCAACTTCCCCTCCGACGGCCCCGTAATG
CAGAAGAAGACCATGGGCTGGGAGGCCTCCTCCGAGCGGATGTACCCCGAGGACGGCGCC
CTGAAGGGCGAGATCAAGCAGAGGCTGAAGCTGAAGGACGGCGGCCACTACGACGCTGAG
GTCAAGACCACCTACAAGGCCAAGAAGCCCGTGCAGCTGCCCGGCGCCTACAACGTCAAC
ATCAAGTTGGACATCACCTCCCACAACGAGGACTACACCATCGTGGAACAGTACGAACGC

[chemical structure]

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A genetically engineered cell line comprising a plurality of cells transformed with at least one polynucleotide encoding a polypeptide, wherein the expressed polypeptide transports an Omega-3 fatty acid across the cell membrane.

2. The genetically engineered cell line of claim 1, wherein the polynucleotide comprises DNA, RNA, or a fragment thereof.

3. The genetically engineered cell line of claim 1, wherein the polynucleotide comprises a synthetic polynucleotide.

4. The genetically engineered cell line of claim 1, wherein the polynucleotide is about 90%, 91%, 92%, 93%, 94%, 95%, 96%, 97%, 98%, 99%, 100% identical to SEQ ID 1.

5. The genetically engineered cell line of claim 1, wherein the polynucleotide is about 90%, 91%, 92%, 93%, 94%, 95%, 96%, 97%, 98%, 99%, 100% identical to SEQ ID 2.

6. The genetically engineered cell line of claim 1, wherein the polynucleotide stably integrates into the genome of the cell.

7. The genetically engineered cell line of claim 1, wherein the plurality of cells comprise skin fibroblasts, adipose tissue stem cells, primary retinal pigment epithelial cells, human adult stem cells, transdifferentiated neuronal cells, pericytes, and macrophages.

8. The genetically engineered cell line of claim 1, wherein the plurality of cells are isolated from skin, adipose tissue, bone marrow, blood, brain tissue, or ocular tissue.

9. The genetically engineered cell line of claim 1, wherein the polypeptide comprises Adiponectin receptor 1 or a fragment thereof; membrane-type Frizzled Related Protein or a fragment thereof; or a combination thereof.

10. The genetically engineered cell line of claim 1, wherein the polypeptide is about 90%, 91%, 92%, 93%, 94%, 95%, 96%, 97%, 98%, 99%, 100% identical to SEQ ID NO: 3 or SEQ ID NO: 4.

11. The genetically engineered cell line of claim 1, wherein the polypeptide is about 90%, 91%, 92%, 93%, 94%, 95%, 96%, 97%, 98%, 99%, 100% identical to SEQ ID NO: 5, SEQ ID NO: 6, or SEQ ID NO: 7.

12. The genetically engineered cell line of claim 1, wherein the omega-3 fatty acid comprises docosahexaenoic acid (DHA) and eicosapentaenoic acid (EPA).

13. A method of generating the genetically engineered cell line of any one of claims 1-12, the method comprising:
obtaining a plurality of cells;
introducing into the plurality of cells at least one polynucleotide encoding a polypeptide, wherein the expressed polypeptide transports an Omega-3 fatty acid across the cell membrane.

14. The method of claim 13, further comprising detecting the presence of the polypeptide within the plurality of cells.

15. The method of claim 14, wherein detecting comprises FACS or immunohistochemistry.

16. The method of claim 13, wherein the polynucleotide comprises DNA, RNA, or a fragment thereof.

17. The method of claim 13, wherein the polynucleotide comprises a synthetic polynucleotide.

18. The method of claim 13, wherein the polynucleotide is about 90%, 91%, 92%, 93%, 94%, 95%, 96%, 97%, 98%, 99%, 100% identical to SEQ ID 1.

19. The method of claim 13, wherein the polynucleotide is about 90%, 91%, 92%, 93%, 94%, 95%, 96%, 97%, 98%, 99%, 100% identical to SEQ ID 2.

20. The method of claim 13, wherein the polypeptide comprises Adiponectin receptor 1 or a fragment thereof; membrane-type Frizzled Related Protein or a fragment thereof; or a combination thereof.

21. The method of claim 13, wherein the polypeptide is about 90%, 90%, 91%, 92%, 93%, 94%, 95%, 96%, 97%, 98%, 99%, 100% identical to SEQ ID NO: 3 or SEQ ID NO: 4.

22. The method of claim 13, wherein the polypeptide is about 90%, 90%, 91%, 92%, 93%, 94%, 95%, 96%, 97%, 98%, 99%, 100% identical to SEQ ID NO: 5, SEQ ID NO: 6, or SEQ ID NO: 7.

23. The method of claim 13, wherein the Omega-3 fatty acid comprises docosahexaenoic acid (DHA).

24. The method of claim 13, wherein the plurality of cells comprise stem cells, skin fibroblasts, adipose tissue stem cells, primary retinal pigment epithelial cells, human adult stem cells, transdifferentiated neuronal cells, pericytes, or macrophages.

25. The method of claim 13, wherein the plurality of cells are isolated from adipose tissue, bone marrow, blood, brain tissue, or ocular tissue.

26. A therapeutic composition comprising a plurality of genetically engineered cells of any one of claims 1-12 and a pharmaceutically acceptable carrier.

27. The therapeutic composition of claim 26 wherein the composition further comprises at least one omega-3 fatty acid.

28. The therapeutic composition of claim 27, wherein the omega-3 fatty acid comprises docosahexaenoic acid (DHA) and EPA.

29. A conditioned media possessing the biological property of being neuroprotective, wherein the conditioned media is characterized by being the product of culturing a plurality of genetically engineered cells of any one of claim 1-12 in a media for a period of time, wherein the cells secrete into the media neuroprotective factors.

30. The conditioned media of claim 29, wherein the cells are removed from the media after the culturing.

31. The conditioned media of claim 29, wherein the neuroprotective protective factors comprise at least one cytokine, lipid mediator, exosome, or a combination thereof.

32. The conditioned media of claim 31, wherein the cytokine comprises IL10.

33. The conditioned media of claim 31, wherein the lipid mediator comprises neuroprotection D1 (NPD1), an elovanoid, a docosanoid, or a combination thereof.

34. The conditioned media of claim 31, wherein the exosomes are derived from stem cells.

35. The conditioned media of claim 29, wherein the period of time comprises about 1, 2, 3, 4, 5, 6, 7, 8, 9, 10, or greater than 10 days.

36. The conditioned media of claim 29, wherein the media is neuroprotective.

37. A method of treating a subject afflicted with a neurological disease, the method comprising administering to the subject any one of the compositions of claim 26-36.

38. A method of reducing or ameliorating symptoms associated with a neurological disease, the method comprising administering to the subject any one of the compositions of claim 26-36.

39. A method of reducing or ameliorating pathogenesis associated with a neurological disease, the method comprising administering to the subject any one of the compositions of claim 26-36.

40. A method of delaying the onset and/or progression of a neurological disease, the method comprising administering to a subject any one of the compositions of claims 26-36.

41. A method of promoting neuronal recovery and restoration of function the method comprising administering to the subject any one of the compositions of claim 26-36.

42. The method of any one of claims 37-41, further comprising administering to the subject an Omega-3 fatty acid.

43. The method of claim 42, wherein the Omega-3 fatty acid comprises Docosahexaenoic acid.

44. The method of any one of claims 37-41, wherein at least one of the plurality of genetically engineered cells engrafts within a tissue of the nervous system of the subject.

45. The method of claim 44, wherein the nervous system comprises the central nervous system, the peripheral nervous system, or combination thereof.

46. The method of claim 44, wherein the tissue comprises the brain, spinal cord, optic nerve or combination thereof.

47. The method of any one of claims 37-41, wherein the neurological disease comprises a disease associated with neuroinflammation, neuronal cell death, neuronal cell injury, or a combination thereof.

48. The method of claim any one of claims 37-41, wherein the neurological disease comprises a neurodegenerative disease.

49. The method of any one of claims 37-41 wherein the disease comprises Alzheimer's disease, epilepsy, Parkinson's disease, stroke, Huntington's disease, traumatic brain injury, spinal cord injury, Amyotrophic lateral scelerosis, and age-related macular degeneration.

50. The method of any one of claim 39, wherein pathogenesis comprises neuroinflammation, neuronal death, neuronal injury, Aβ formation, infarct volume, or oxidative stress.

51. The method of any one of claims 37-41, wherein the composition promotes neuronal survival, neuronal differentiation, neurogenesis, neurite growth, synaptogenesis, synaptic protein expression, synaptic function, or a combination thereof.

52. The method of claim 51, wherein neurogenesis is detected using 5-bromo-2′-deoxyuridine, Ki-67, DCX, NeuN, or a combination thereof.

53. A kit comprising a plurality of genetically engineered cells of any one of claims 1-12, the therapeutic compositions of claims 26-28, the conditioned media of claims 29-36, or a combination thereof, and instructions for use.

54. The kit of claim 53, wherein the plurality of genetically engineered cells, the therapeutic compositions, the conditioned media, or a combination thereof are provided in a vessel.
